(19) United States
(12) Patent Application Publication
(10) Pub. No.: US 2005/0010069 A1
(43) Pub. Date: Jan. 13, 2005

(54) Oil ozonolysis

(76) Inventors: Colin Stanley Fitchett, Cambridge (GB); Nicholas Geoffrey Laughton, Cambridge (GB); Colin Graham Chappell, Essex (GB); Mohammed Lokman Khan, Sheffield (GB); Viacheslav Tverezovskiy, Gweynedd (GB); Jeremy Tomkinson, JT (GB); Paul Fowler, Gwynedd (GB)

Correspondence Address: HELLER EHRMAN WHITE & MCAULIFFE LLP, 1666 K STREET,NW, SUITE 300, WASHINGTON, DC, 20006, US

(21) Appl. No.: 10/497,572

(22) PCT Filed: Dec. 11, 2002
(86) PCT No.: PCT/GB02/05610

(30) Foreign Application Priority Data
Dec. 11, 2001 (GB) 0129590.6

(51) Int. Cl. C07C 27/00

(52) U.S. Cl. 568/959

(57) ABSTRACT

Ozonolysis is a well known process involving reacting ozone with alkene compounds, for example in unsaturated vegetable oils or free fatty acids and esters thereof, to form ozonolysis products (e.g. ozonides). The invention concerns ozonolysis of unsaturated oils (e.g. unsaturated plant oils and/or unsaturated animal oils) in the presence of a participating co-reactant to form reaction products particularly suitable for use in the formation of resins.

The present invention relates to ozonolysis of unsaturated oils (e.g. unsaturated plant oils and/or unsaturated animal oils) to form reaction products particularly suitable for use in the formation of resins.

Ozonolysis is a well known process involving reacting ozone with alkene compounds, for example in unsaturated vegetable oils or free fatty acids and esters thereof, to form ozonolysis products (e.g. ozonides). Reductive decomposition of the ozonolysis products results in various compounds such as aldehydes (see Pryde, E. H. et al. (1961) J. Am. Oil Chemists' Soc. 38: 375-379).

Aldehydes and other compounds derived from reductive decomposition of ozonolysis products are valuable in the formation of resins and various polymeric materials. Pryde et al. (1961; supra) disclose reacting aldehyde mixtures formed during ozonolysis with phenol to form resins. WO 00/78699 discloses ozonolysis of unsaturated oil to form aldehyde and/or peroxide resin precursors. WO 00/31015 teaches ozonolysis of cashew nut shell liquid (CNSL) followed by reduction of the ozonolysis products to form a mixture having phenolic components and aldehydes, the mixture suitable for use as a binder in the formation of composite products.

To improve the dissipation of exothermic heat from the ozonolysis of unsaturated oils, the reaction has conventionally been conducted at lowered temperatures (for example about −20° C. to 14° C.) and in an excess of solvent solution. The prior art teaches that use of excess solvents is necessary to improve the mixing of the unsaturated oils and ozone, and to reduce the formation of unwanted polymers and other byproducts that may result from local overheating. Many of the effective organic solvents used (for example lower aliphatic alcohols, liquid hydrocarbons such as hexane, or chlorinated solvents such as methylene chloride) are hazardous because they have high vapour pressures, are flammable, and may be toxic.

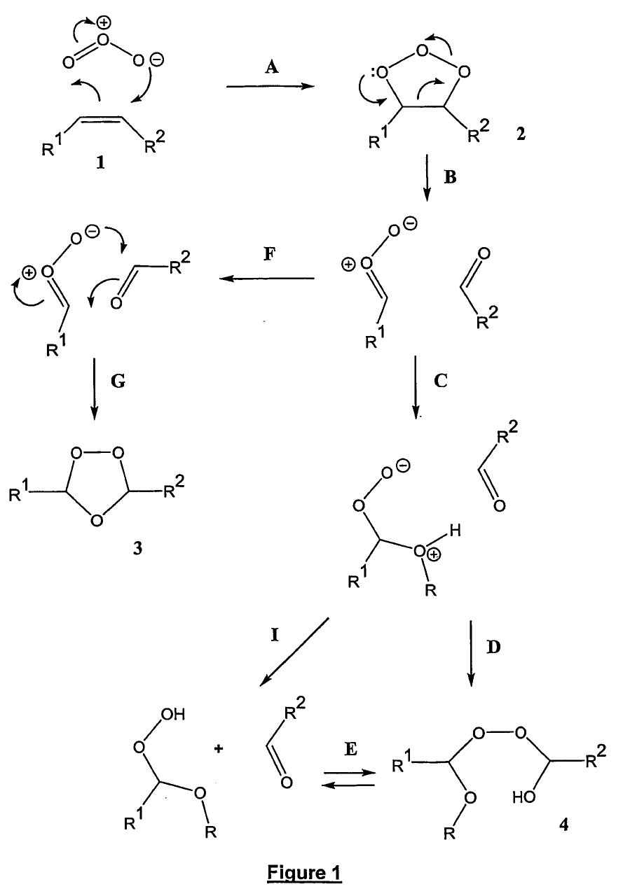

Solvents may be classified broadly as “participating” or “non-participating” solvents. Participating solvents will react chemically with ozonide intermediates formed during the ozonolysis reaction. For example, the prior art teaches that ozonolysis in a participating solvent which is protic (ie. capable of donating a proton) such as an alcohol or water will lead to formation of a hydroperoxide, whereas ozonolysis in aprotic, non-participating solvents such as hydrocarbons (e.g. cyclohexane, hexane) and chlorinated hydrocarbons (e.g. dichloromethane and chloroform) will lead to the formation of ozonides (see FIG. 1, described infra).

The use of water as a vehicle for ozonolysis of unsaturated fatty acids, and subsequent oxidation to form dibasic and monobasic acids, is taught in U.S. Pat. No. 2,865,937. The two step process involves low temperature ozonolysis (preferably in the range 15° C. to 30° C.) of the unsaturated fatty acid in an amount of water approximately one to six times by mass of the unsaturated fatty acid and solvent (such as caproic acid) to form ozonides, followed by high temperature oxidative decomposition of the ozonides (at temperatures of 100° C. to 150° C.).

However, it has been reported in U.S. Pat. No. 3,504,038 that when the ozonolysis step disclosed in U.S. Pat. No. 2,865,937 was repeated using vegetable oils such as linseed oil or soybean oil, an extremely thick, creamy water-in-oil layer was formed, resulting in insufficient mixing with ozone and the formation of unwanted by-products, and in reduced yields of aldehyde products after reduction. In U.S. Pat. No. 3,504,038 this problem was addressed by mixing vegetable oil with a straight chain saturated aldehyde and combining this mixture with water as a solvent (in quantities 2.2 to 2.7 parts by mass of water per part of vegetable oil) prior to ozonolysis at ambient temperatures (about 23° C. to 38° C.).

The present inventors disclose a novel improved process for the ozonolysis of unsaturated oils.

According to the present invention there is provided a process for the ozonolysis of unsaturated oils to form ozonolysis reaction products, comprising reacting together ozone, unsaturated oil and a participating co-reactant, wherein the participating co-reactant is in 0.01 to less than 1 part by mass per part of the unsaturated oil.

The process according to the present invention utilises a participating co-reactant present in insufficient quantities to be deemed a solvent. This optimises the formation of product but minimises the formation of unwanted by-products. It also allows the reaction to be heated to levels previous not possible in the prior art, as there is no excess solvent which in the prior art reacts with ozone to form unwanted by-products or is simply hazardous at elevated temperatures. In contrast to the prior art, the ozonolysis reaction products according to the present invention do not require immediate reduction or hydrogenation to yield a useful product.

The terms “ozonization” and “ozonation” are used interchangeably herein, each meaning reacting with ozone.

The ozonolysis reaction products may essentially comprise peroxy hemi-acetals. For example, the peroxy hemi-acetal may be a 1-(1-alkoxyalk-1-yl peroxy)-alk-1-ol when an alkyl (such as an alcohol) is the participating co-reactant, and a 1-(1-hydroxyalk-1-yl peroxy)-alk-1-ol where water is the participating co-reactant.

As discussed further in the experimental section below, the formation by ozonolysis of peroxy hemi-acetyl compounds per se is known. The present invention provides in one aspect an improved process for forming these compounds.

The participating co-reactant may comprise water and/or an alcohol (for example: ethanol, industrial methylated spirits or isopropanol). Alternatively, the participating co-reactant may be any or a mixture of water or an alcohol (for example: ethanol, industrial methylated spirits or isopropanol). The participating co-reactant is preferably a protic co-reactant, for example an alcohol and/or water, but may be an aprotic co-reactant, for example ketones (e.g. acetone), esters, aldehydes, phenols, amines and/or thiols. Mixtures of these participating co-reactants may be used.

The process may comprise introduction of the ozone into a reactor vessel containing a mixture comprising the unsaturated oil and the participating co-reactant. This process may be conducted at a temperature of −5° C. to 100° C., preferably 15° C. to 60° C.

Alternatively, the process may comprise introducing into a reactor vessel containing the unsaturated oil a vapour stream comprising the participating co-reactant and the ozone. In this embodiment, the process may be conducted at a temperature of 10° C. to 140° C.

In a further embodiment, the process may comprise introducing into a reactor vessel containing the ozone a mixture comprising the unsaturated oil and the participating co-reactant. Here, the process may be conducted at a temperature of −5° C. to 100° C., preferably 15° C. to 50° C.

In yet another embodiment, the process may comprise introducing separately into a reactor vessel: a spray comprising the unsaturated oil; the ozone; and a vapour stream comprising the participating co-reactant. This process may be conducted at a temperature of 70° C. to 140° C.

The process may be a batch process or a continuous process.

The ozone may be present in 0.1 to 0.6 parts by mass per part of unsaturated oil and participating co-reactant. The ozone may be present at a concentration of 1-15% by mass in a mixture with air or oxygen.

The end point for ozonolysis can be judged using thin layer chromatography (TLC), high performance liquid chromatography (PLC), gas chromatography—mass spectrometry (GC-MS) or chemical methods such as the starch iodide test. Such tests may be used to check periodically for the end point of the ozonolysis, i.e. when none of the unsaturated oils present in the starting material are present in the reaction mixture.

Alternatively, the reaction could be terminated prior to the end point for ozonolysis if partial ozonolysis reaction products are required. Ozone is relatively expensive, so it may be desirable to terminate ozonolysis prior to completion and harvest the products formed at termination. The methods mentioned above may thus also be used to analyse the progress of a reaction to determine whether desired products have been formed.

The unsaturated oil may comprise plant oils such as vegetable oil, for example cashew nut shell liquid (CSNL). Plant oils include any unsaturated oil that is derived from plant material (e.g. tri-, di-, mono-glycerides, free fatty acids etc.). The present invention can be practised using isolated or purified/semi-purified oils extracted from a suitable plant source. However, in addition, or alternatively, the oil bearing plant tissues (preferably suitably pre-treated, e.g. comminuted) can be subjected to ozonolysis to produce a product comprising plant matter containing oxidative cleavage products. Plant oils useful in forming the products of the invention include unsaturated plant oils such as tung oil, mono-, di-, and tri-glyceride oils such as oils from oil seed rape, linseed, soya, olive oil, castor oil, mustard seed oil, ground nut oil, and phenolic oils such as cashew nut shell liquid (CNSL).

The unsaturated oil may comprise unsaturated animal oils, for example fish oils and/or fractionated tallow.

The process may further comprise a heat-treating step following ozonolysis. During this heat-treating step, the ozonolysis reaction products may be degassed, allowing for example gaseous CO₂ and/or gaseous O₂ to be removed. The heat-treating step may involve heating to about 80° C. or above, preferably about 80°-125° C. or about 80° C.-110° C. or about 80° C.-100° C. or about 80° C.-90° C. Once the reaction mixture has been heated to these temperatures, the reaction may be exothermic and self-sustaining, no longer requiring the addition of further heat.

Further provided according to the present invention is a process for making an adhesive-forming compound (e.g. an aldehyde), comprising treating under reducing conditions the ozonolysis reaction products formed by the process defined above.

Reduction of the ozonolysis reaction products (e.g. ozonides) can be carried out using any of a variety of reducing conditions. Thus, reduction can be effected using a suitable metal, such as a transition metal (e.g. zinc), preferably in the presence of an acid.

For example, formation of the adhesive forming compound under reducing conditions can, for example, be carried out in the presence of zinc and acetic acid. Alternatively, other methods (e.g. standard methods) of achieving reducing conditions can be used and examples of such methods include catalytic hydrogenation in the presence of a metal catalyst such as a transition metal catalyst: e.g. hydrogen may be bubbled through the reaction mixture in the presence of a catalyst such as Pd—C (catalytic palladium hydroxide on calcium carbonate). Other reducing agents that can be used include iodide (e.g. sodium, potassium, calcium etc)+acetic acid; dimethyl sulphide; thiourea; triphenyl phosphine; trimethyl phosphate and pyridine.

A further alternative, and particularly preferred, reducing agent is a reducing sugar. The reducing sugar can be for example a monosaccharide or a disaccharide, and can be an aldose or a ketose sugar. Examples of reducing sugars are hexose monosaccharide sugars such as glucose, mannose, allose, and galactose, and disaccharides such as maltose. A presently preferred sugar is alpha-D-glucose.

Also provided is a process for forming an adhesive material, comprising treating with an acidic material (an “acid catalyst”) the adhesive-forming compound made by the above process. Examples of acid catalysts include sulphonic acids, particularly substituted sulphonic acids such as aromatic sulphonic acids, e.g. p-toluenesulphonic acid. Alternatively, the process for forming an adhesive material may comprise treating with a basic material (a “base catalyst”) the adhesive-forming compound made by the above process. Either or both of these processes for forming an adhesive material may further comprise a heat-treating step. The heat-treating step may involve heating to about 80° C. or above, preferably about 80°-125° C. or about 80° C.-110° C. or about 80° C.-100° C. or about 80° C.-90° C.

In another embodiment there is provided a process for forming an adhesive material, comprising heat-treating the adhesive-forming compound made by the above process.

The adhesive material formed by the process may be a resin. Also provided is a resin derived from this process.

Further provided is a resin derived from ozonolysis reaction products as defined above.

The resin may be a cured thermosetting resin.

The resins of the invention have a large number of applications, and examples of uses of the resins are in the formation and manufacture of moulded panels, non-woven materials, fibre-glass products, boards, paper treatments, fabric treatments, spun textiles, toys (e.g. children's toys), lubricants, adhesives, castings, automotive components (such as bumpers, fenders, steering wheels, interior panels and mouldings, exterior trim and mouldings), upholstery (as padding or mouldings), binding recycled materials, foundry castings and casting materials (for example binders for refractory articles), bearings, films and coatings, packaging, foams, paint components, pipes, architectural and building products such as door and window frames, varnishes, release controlling coatings such as release controlling coatings for pharmaceuticals, solid prosthetic devices and medical devices, and wood treatment agents, e.g. for preserving and modifying the properties of wood.

Articles of the type listed above, formed from resins derived from an ozonolysis reaction product formed by the process described herein represent a further aspect of the invention.

An apparatus for performing the process is also provided according to the present invention, as described below. In a further embodiment, the apparatus comprises a spray system. This would be analogous to a paint spray system, where the oil, ozone and co-reactant are mixed together at a dispensing device such as a nozzle and sprayed into a tank, ensuring intimate mixing of the reactants. Water as a co-reactant may be delivered as steam or, preferably, as atomised water vapour.

Further provided is a solid composite material (“composition”) comprising a resin as described herein.

The compositions of the invention can be cured in a variety of different ways. For example, the compositions are capable of undergoing self-crosslinking through a range of chemistries. The properties of the resulting cured resins or compositions are influenced by the molecular size of the compounds making up the oxidative cleavage product and the number of reactive sites, both being determined by the chain length of the starting material and the degree of unsaturation.

Thus, for example, for aldehyde adhesive-forming compounds, crosslinking mechanisms include condensations (e.g. aldol condensations), aldehyde polymerisations, and polymerisation reactions with residual reducing sugars e.g. glucose.

For hydro-peroxide adhesive-forming compounds, polymerisation can take place with residual olefin bonds within the oxidative cleavage products, or by means of homocross-linking of peroxide or alkyl peroxide moieties.

Curing of the compositions can also be effected by the formation of heteropolymers, for example with compounds such as amines or phenols having free amino or hydroxyl groups, or other nucleophiles.

Heteropolymer coupling partners (e.g. co-monomers) can be incorporated either during the preparation of the adhesive-forming compounds or at the curing stage. Suitable species are generally nucleophiles that can cross-link and become incorporated into the resin structure. Such heteropolymers have modified properties resulting from changes to the crosslinking sites and molecular size of the precursors. Useful properties that can be controlled by the choice of additive include: elasticity, rigidity, brittle fracture, toughness, shrinkage, resistance to abrasion, permeability to liquids and gases, UV resistance and absorbance, biodegradability, density and solvent resistance.

The properties of the uncured compositions may also be usefully modified using additives to control, for example, the viscosity and flow characteristics of the compositions on a filler surface or through spray jets. Examples of materials that can be added to the compositions of the invention include aromatics, phenol, resorcinol and other homologues of phenol, cashew nut shell liquid (CNSL), lignins, tannins and plant and other polyphenols, proteins such as soy protein, gluten, casein, gelatin, and blood albumin; glycols and polyols such as ethylene glycol, glycerol and carbohydrates (e.g. sugars and sugar alcohols); amines, amides, urea, thiourea, dicyandiamide, and melamine; isocyanates such as MDI; heterocyclic compounds such as furfural, furfuryl alcohol, pyridine and phosphines.

Polymerisation or curing of the compositions and adhesive-forming compounds typically requires a catalyst. Examples of catalysts include acids such as para-toluene sulphonic acid, sulphuric acid, hydrochloric acid and salts that liberate acids, e.g. ammonium sulphate and ammonium hydrochloride. Further examples of catalysts include Lewis acids such as zinc chloride and zinc acetate, aluminium compounds such as aluminium chloride and boron compounds such as boron trifluoride (e.g. in its trifluoroboroetherate form), and alkalis such as sodium and potassium hydroxide. Still further examples of catalysts include radical initiators such as dibenzoylperoxide or AIBN [bis(-azoisobutyronitrile), also known as 2,2′-Azobis(2-methylpropionitrile)].

The invention will be described in further detail below with reference to the accompanying figures. Of the figures:

FIG. 1 Shows a scheme of proposed compounds formed by ozonolysis of unsaturated fatty acids such as oils;

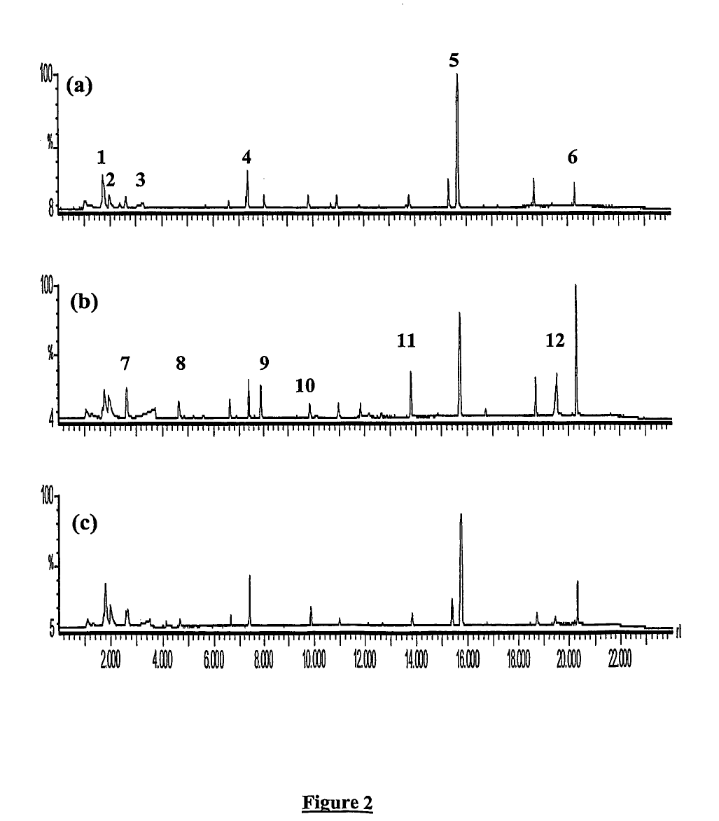

FIG. 2 Shows GC-MS traces from solid phase microextraction (SPME) of (a) rapeseed oil ozonated with a large excess of IPA (simulating prior art) for 30 minutes; (b) the same sample as for (a) but after a further 4 hours of ozonolysis (4.5 hours total); (c) rapeseed oil ozonated with optimised isopropanol (IPA) and ozone. Key peak identities: 1=isopropanol; 2=formic acid; 3=acetic acid; 4=hexanal; 5=nonanal; 6=isopropyl nonanoate; 7=isopropyl formate; 8=isopropyl acetate; 9=octane; 10=heptanal; 11=isopropyl hexanoate; 12=nonanoic acid;

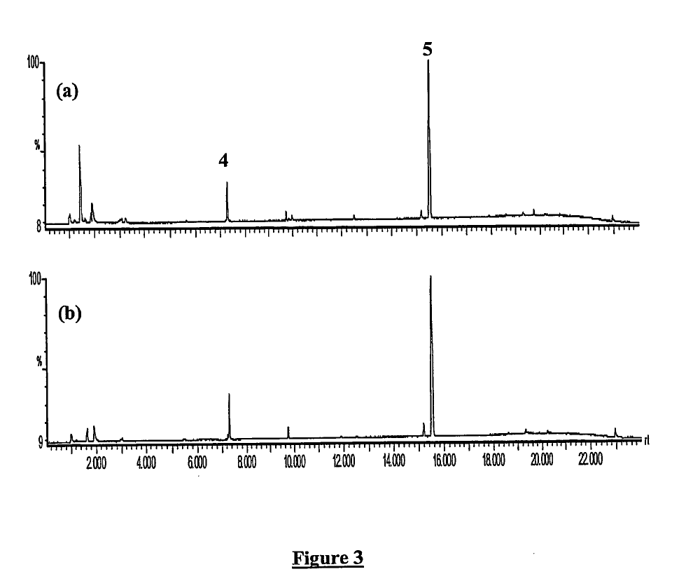

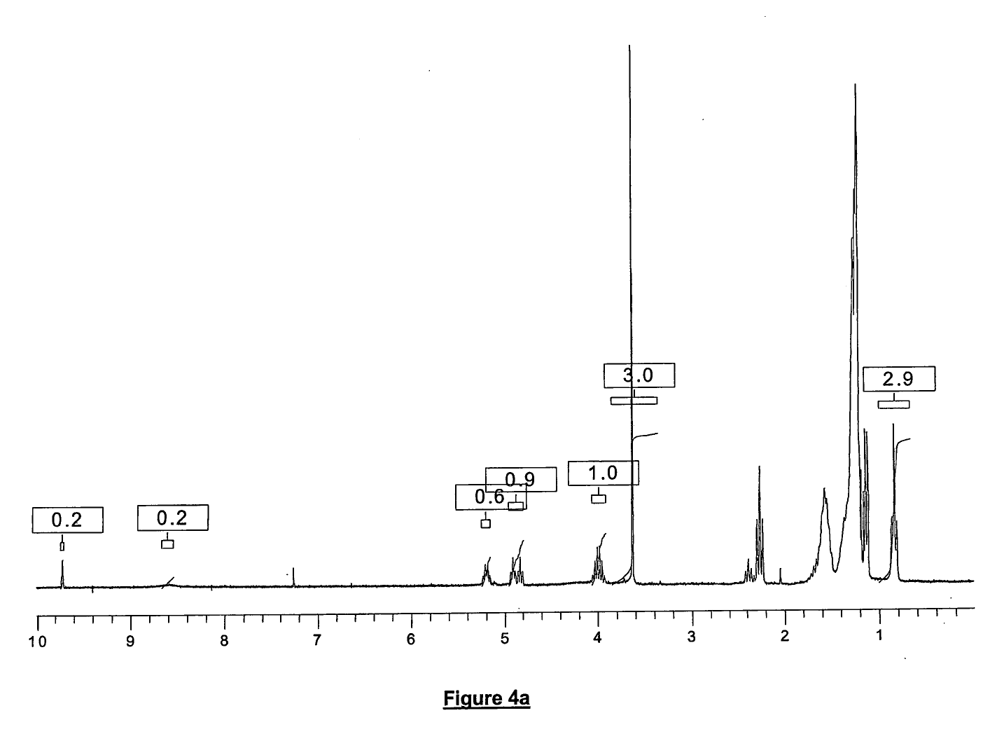

FIG. 3 Shows GC-MS traces from SPME extractions of (a) rapeseed oil ozonated with optimised ozone and with optimised industrial methylated spirits (IMS)/water as the co-reactant, and (b) rapeseed oil with optimised ozone and optimised water alone as the co-reactant. Ester formation is eliminated when water is the co-reactant, and in both cases the overall volatile profile is simpler and less intense than when using excess ozone/co-reactant, with mainly the aldehydes hexanal (4) and nonanal (5) predominating;

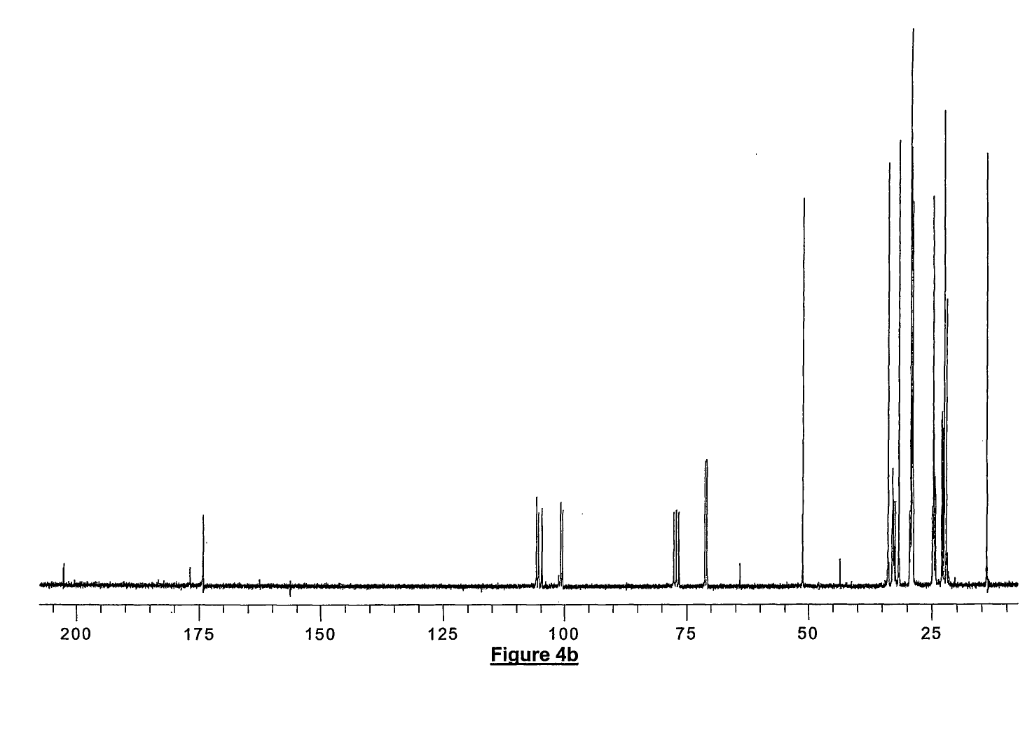

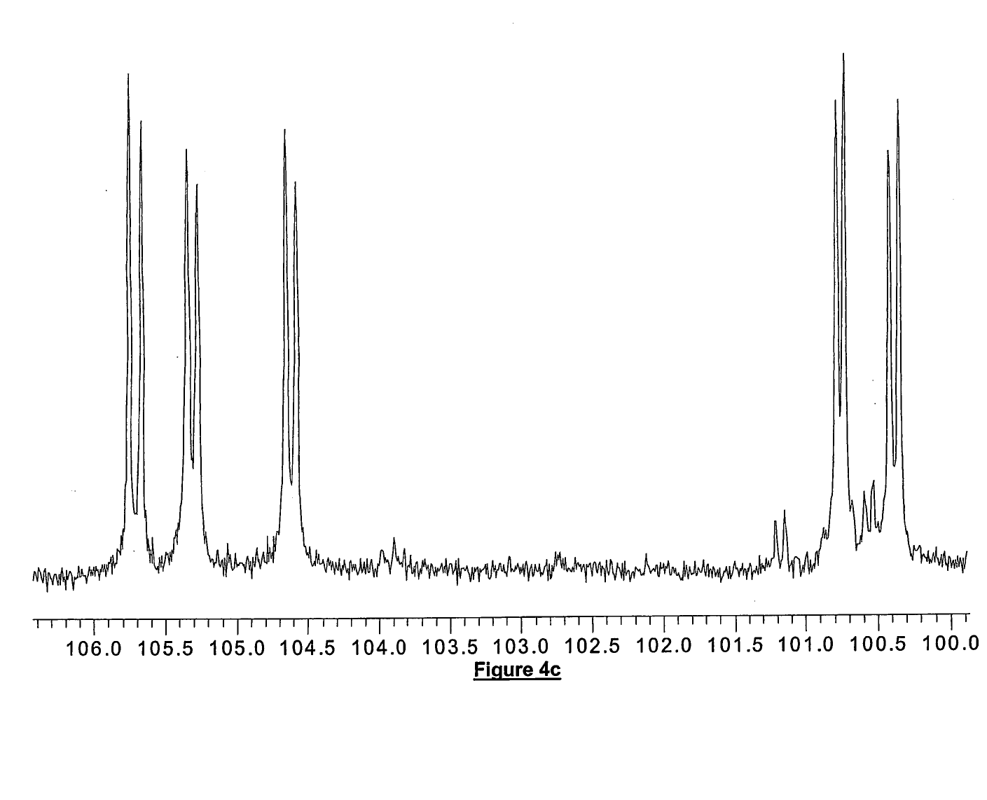

FIG. 4 Shows ¹H NMR (FIG. 4a) and ¹³C NMR (FIG. 4b, with FIG. 4c in more detail over indicated range) of a reaction mixture following ozonolysis of methyl oleate in excess IPA;

FIG. 5 Shows the chemical structures of the main products formed by ozonolysis of methyl oleate in IPA;

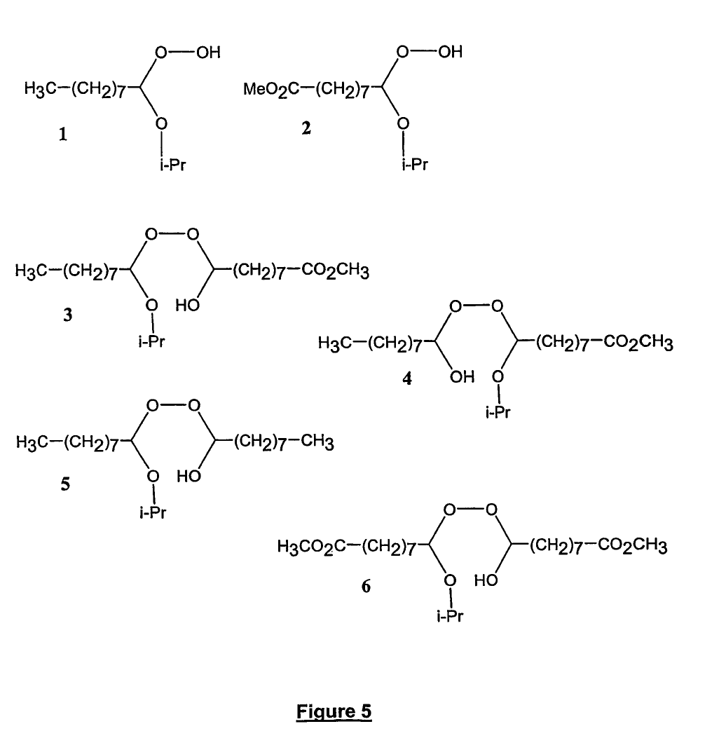

FIG. 6 Shows ¹³C NMR of the reaction products following ozonization of methyl oleate in IPA (1.5 mol. equivalents);

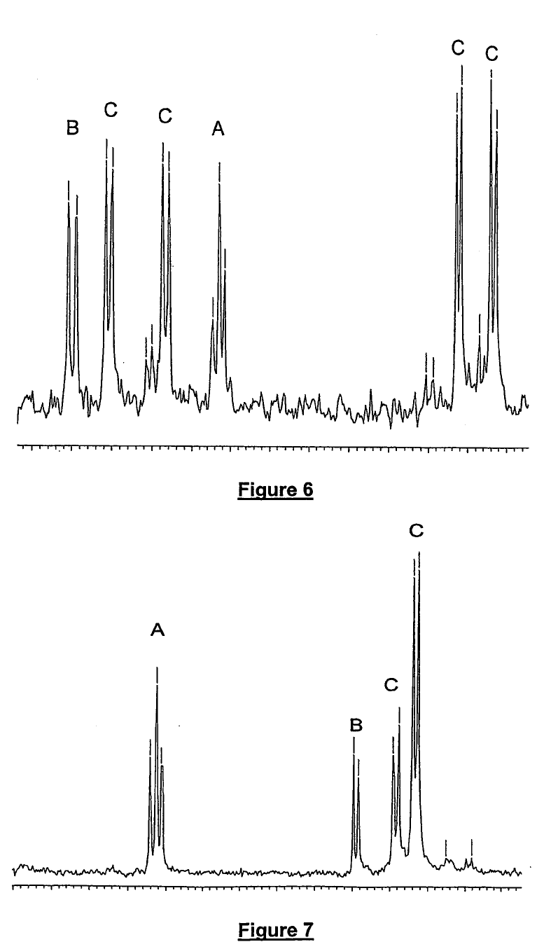

FIG. 7 Shows ¹³C NMR of the reaction products following ozonization of methyl oleate in the presence of water (1.36 mol. equivalents);

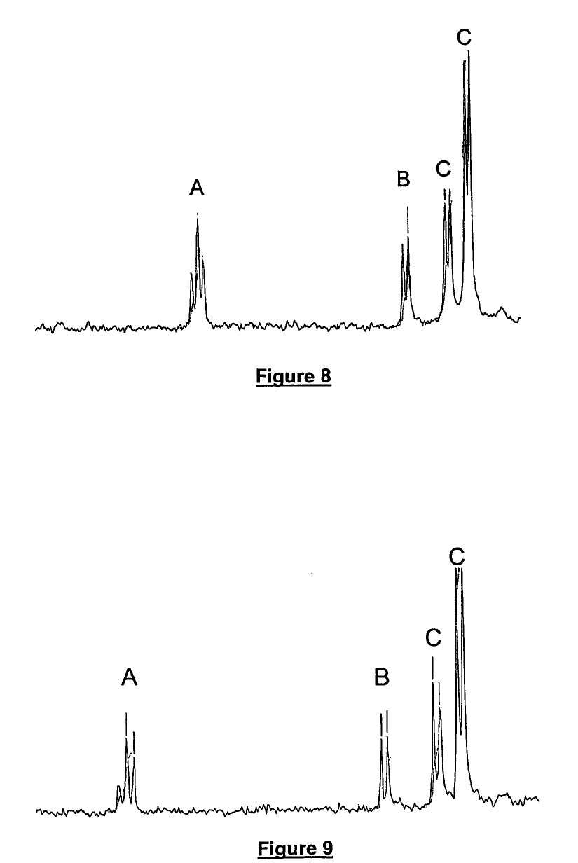

FIG. 8 Shows ¹³C NMR of the reaction products following ozonization of methyl oleate in the presence of excess water (5 mol. equivalents);

FIG. 9 Shows ¹³C NMR of the reaction products following ozonization of methyl oleate in the presence of a large excess of water (28 mol. equivalents);

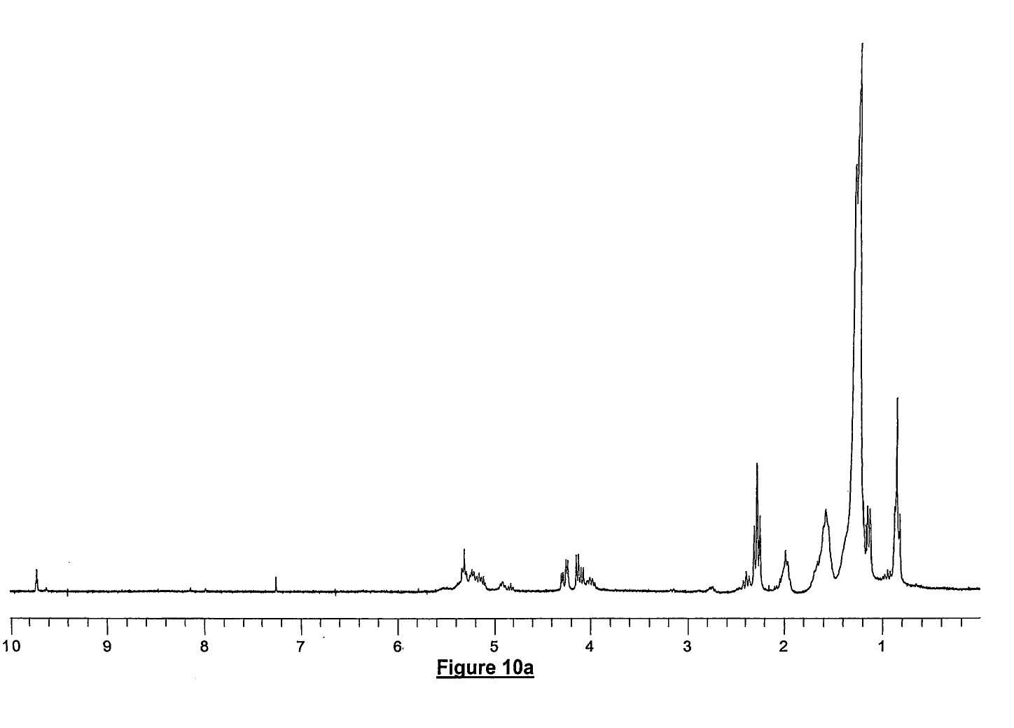

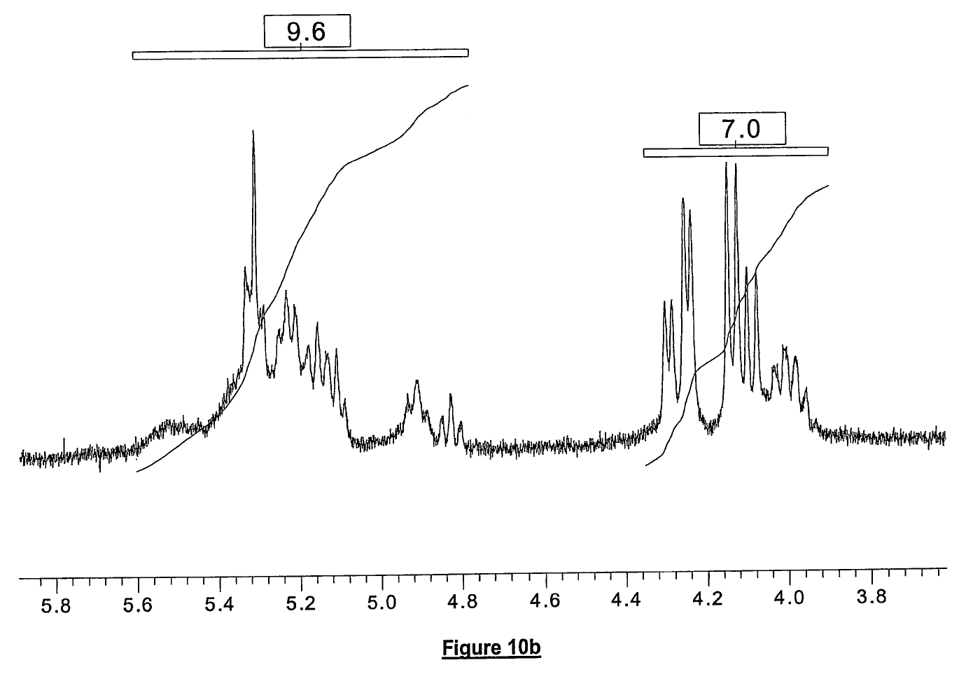

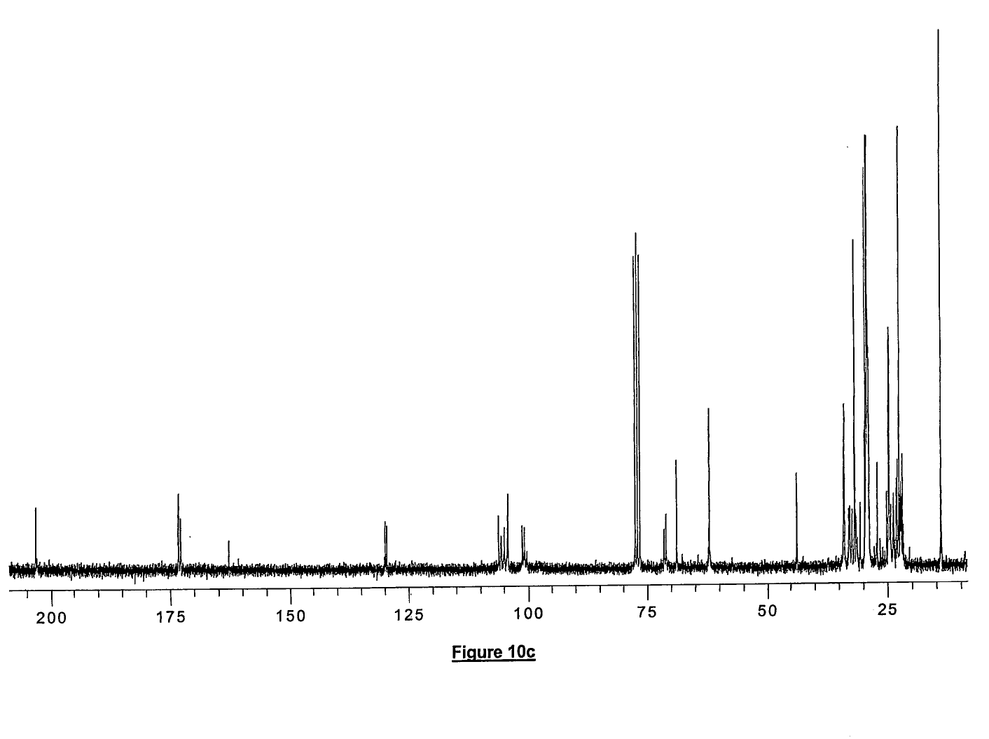

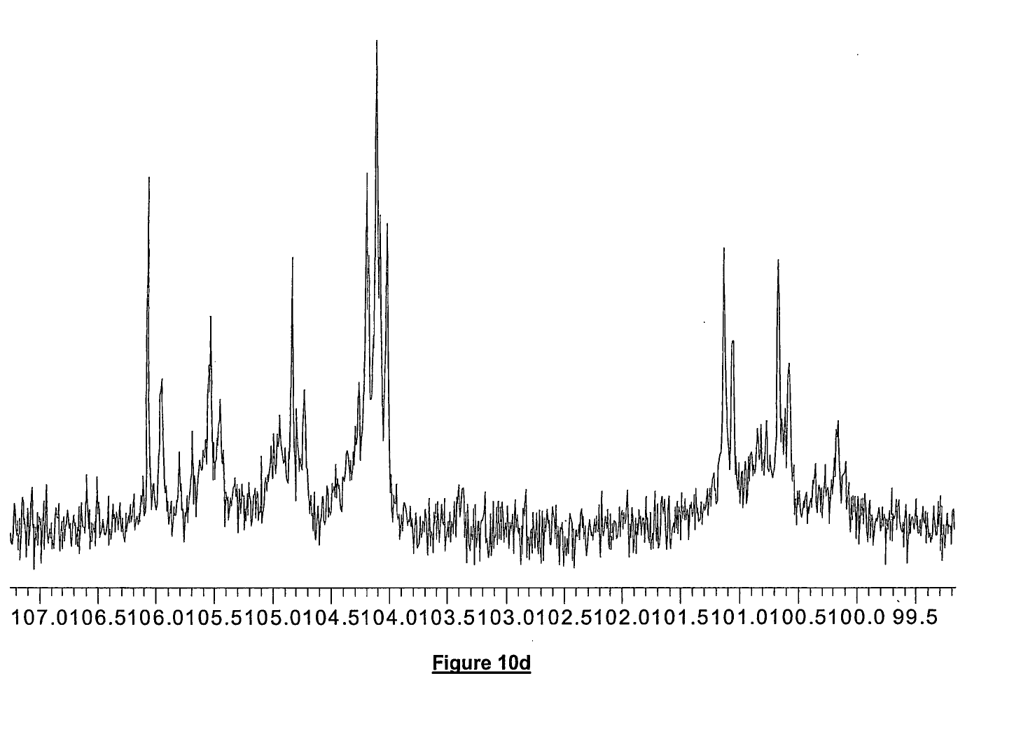

FIG. 10 Shows ¹H NMR (FIG. 10a, with FIG. 10b in more detail over indicated range) and ¹³C NMR (FIG. 10c, with FIG. 10d in more detail over indicated range) of the reaction mixture following ozonolysis of rape seed oil (RSO) in IPA;

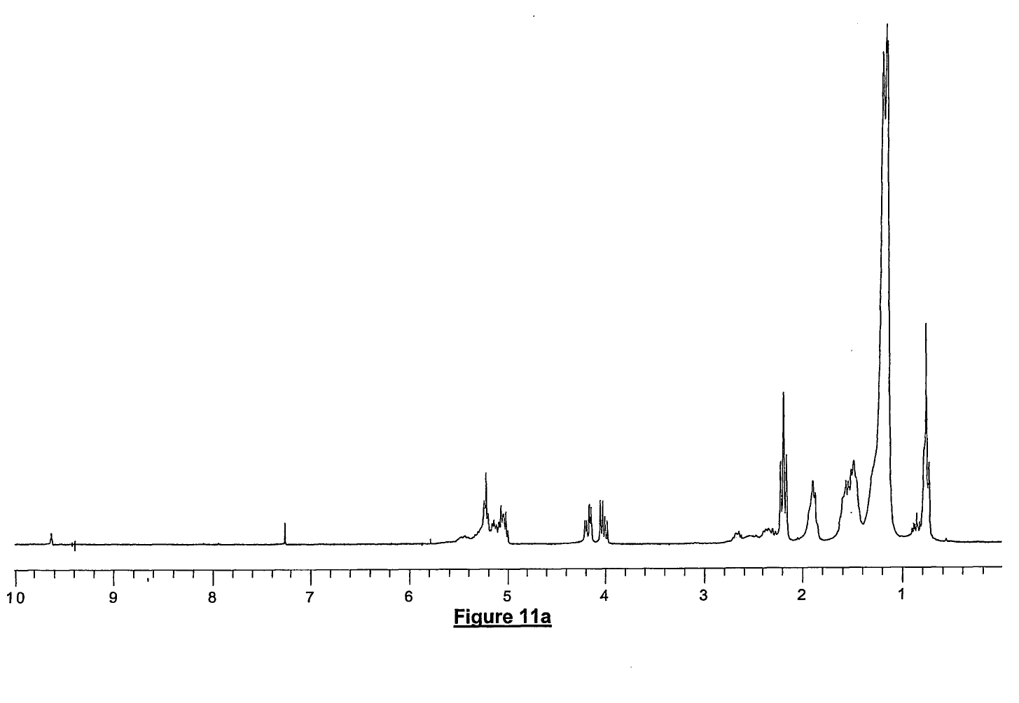

FIG. 11 Shows ¹H NMR (FIG. 11a, with FIG. 11b in more detail over indicated range) of the reaction mixture following ozonolysis of rape seed oil (RSO) in water;

FIG. 12 Shows HPLC chromatograms of partially (FIGS. 12a and 12b) and fully (FIGS. 12c and 12d) ozonized cashew nut shell liquid (CNSL) solutions in isopropyl alcohol/methanol 1:1, with UV detection at 275 nm (FIGS. 12a and 12c) and 254 nm (FIGS. 12b and 12d);

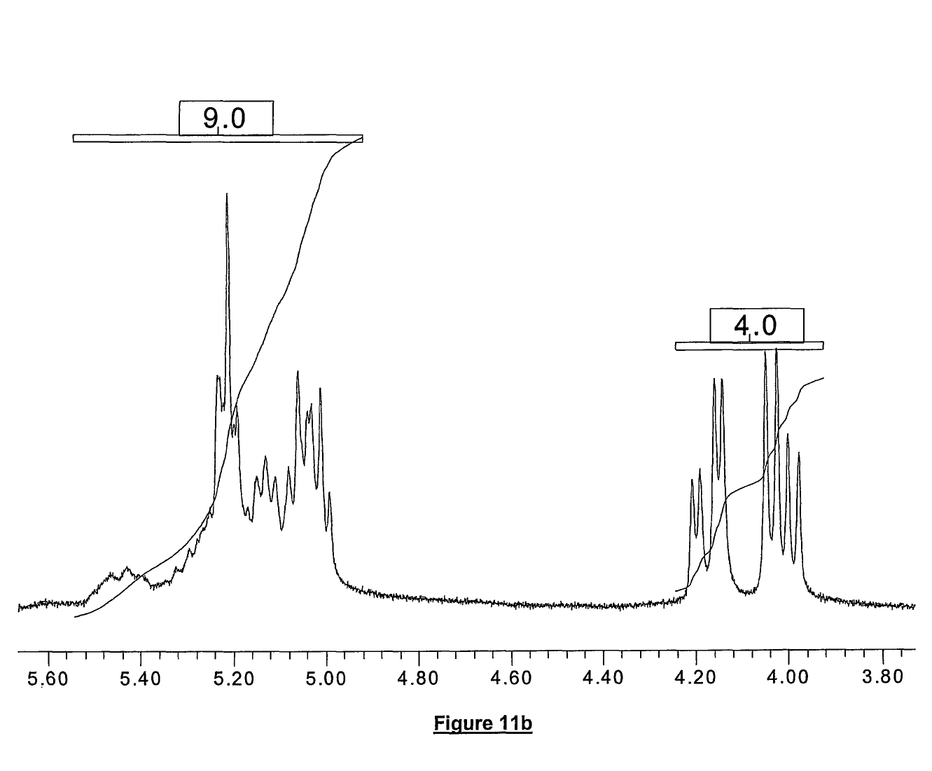

FIG. 13 Is a histogram showing monitoring of ozonization of CNSL by HPLC with UV detection at 254 nm, with the x-axis representing an approximate % ozonization towards the desired end-point of reaction. Peaks 1 and 2 are clusters of compounds which appear as a consequence of ozonization while peaks 3 and 4 are clusters which are compounds present at the start but which are consumed as ozonization proceeds (compare FIG. 12a-d);

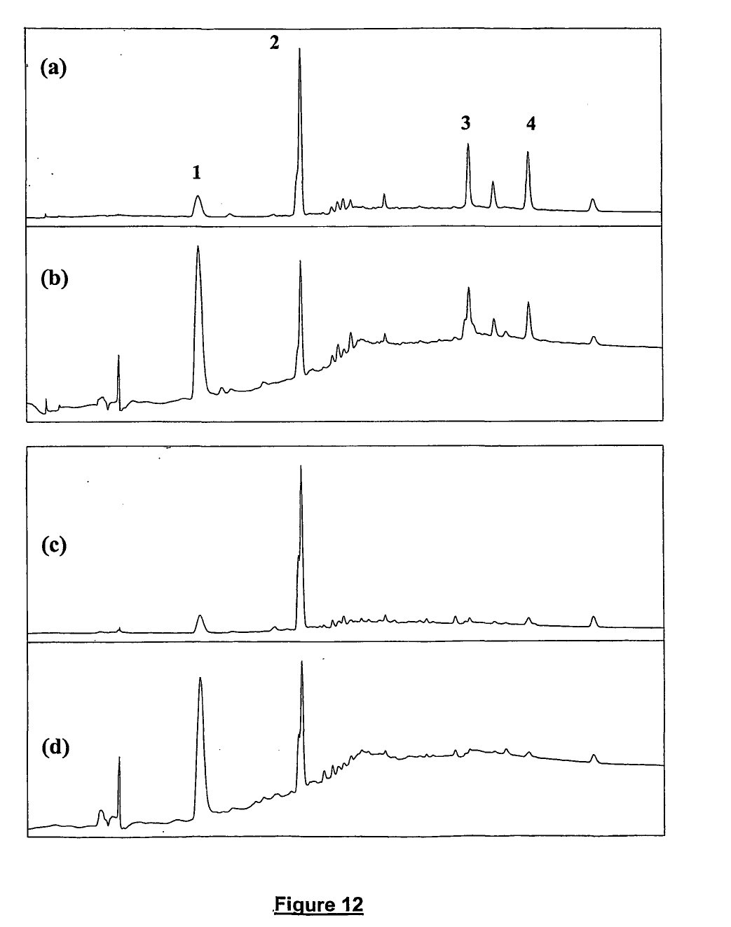

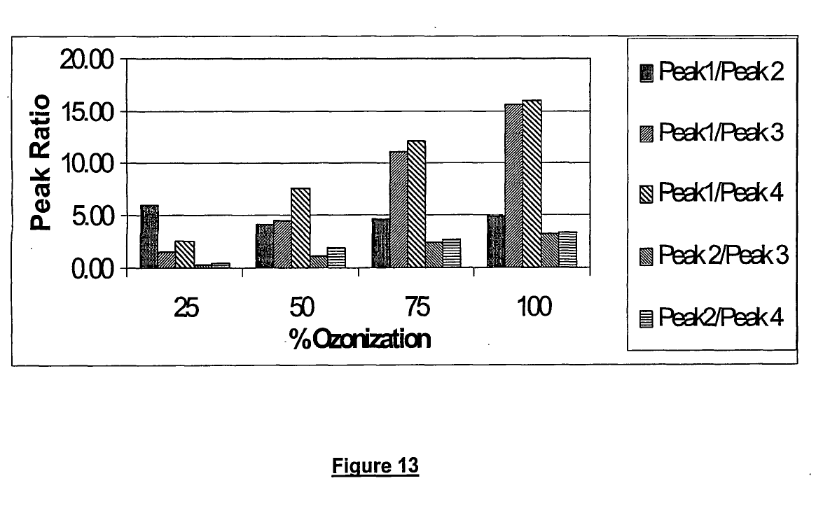

FIG. 14 Shows GC-MS chromatographs of ozonized CNSL with ozonization 25% complete (FIG. 14a), ozonization 50% complete (FIG. 14b) and ozonization complete (FIG. 14c). Peaks 2 and 3 represent unsaturated C7 aldehydes while peak 1 is fully saturated C7 aldehyde (heptanal); and

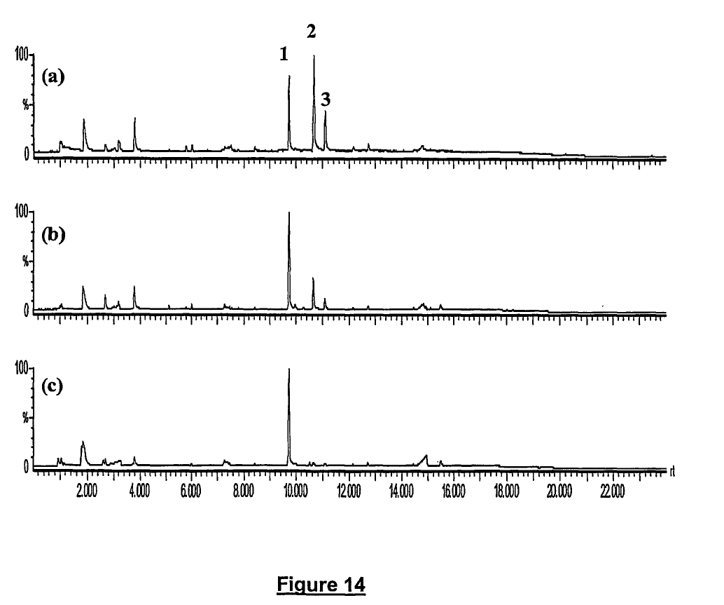

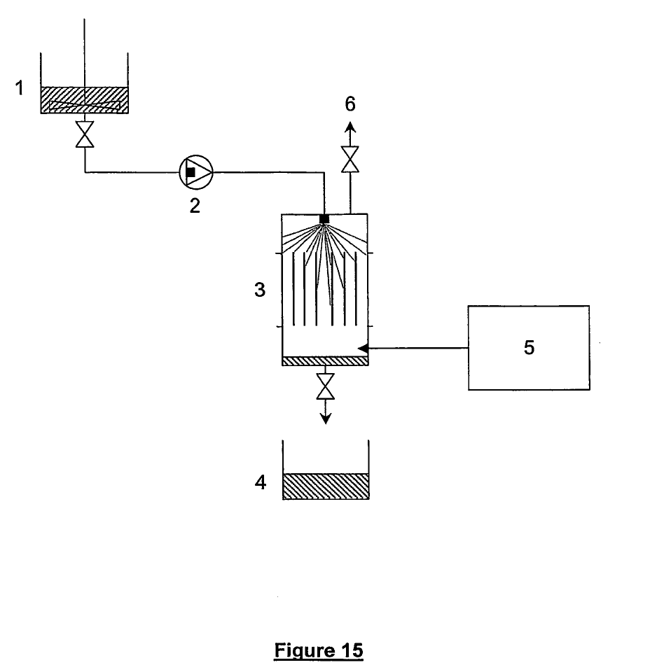

FIGS. 15-19 Illustrate reactor designs for formation of a resin precursor according to the invention.

### EXPERIMENTAL

Overview

For the characterisation of product and co-products, resin precursor samples have been prepared with a range of different raw materials (plant oils refined/unrefined and CNSL) and participating co-reactants (water, ethanol, industrial methylated spirits (IMS), and isopropanol (IPA)). When used in excess, these participating co-reactants act both as solvents and reactants, but in the methodology disclosed herein they serve as reactants only. Our product can be cured subsequently in the presence of acid and a nucleophile to form a thermosetting resin. Persistent volatile organic compounds (VOCs) generated in the bioresin process have been analysed using solid phase microextraction (SPME) and direct injection. The VOCs identified are commonly occurring natural compounds, namely aldehydes and their acetals, acids, alcohols and esters.

Product Composition

The proposed process utilises a participating (e.g. protic) co-reactant such as, but not limited to, water, alcohols (alone or in combination), present in insufficient quantities to be deemed a solvent, to produce peroxy hemi-acetals preferably using cashew nut shell liquid (CNSL) or other vegetable oils (including their free fatty acids and esters thereof) when ozonated. The level of reactants (e.g. protic co-reactant, ozone and unsaturated oil) utilised are optimised for the formation of product but minimise the formation of co-products. The raw material substrate is required to be unsaturated in order for the addition of ozone to occur.

The principal product (resin precursor) of the primary process is a peroxy hemi-acetal as shown in FIG. 1. It is formed sequentially from the addition of ozone across the double bond(s) of the unsaturate (1) to form a primary ozonide (2) as shown in reaction A; followed by cleavage of the primary ozonide (reaction B) to yield an aldehyde and carbonyl oxide which in the presence of a protic solvent (ROH; reactions C and D) re-combine to form a peroxy hemi-acetal (4) (see for example Nishikawa, N. et al., 1995, J. Am. Oil Chemists' Soc. 72: 735-740). This peroxy hemi-acetal (4) forms the main bulk of the resin precursor in the present invention.

The chemistry described above has been verified using methyl oleate standard as the substrate. The product was characterised by GC-MS analysis (which analyses volatiles formed from the reaction product), NR (proton and ¹³C) analysis or HPLC analysis (which analyses non-volatiles of the reaction product), as described further below.

GC-MS Analysis:

Gas Chromatography Parameters:
Gas Chromatograph: HP5890 II
Carrier gas: Helium at 4 psi head pressure
Column: 30 m×0.32 mm I.D. 1.0 μm film thickness DB1 capillary column (ex J+W)
Temperature Programme: 35° C. (hold 4 min) programmed to 75° C. at 20° C./min, then to 130° C. at 5° C./min, then to 250° C. (hold 1 min) at 20° C./min.
Injection: Split (ratio 10:1)
   Mass Spectrometry Parameters:
VG-Trio 1000 quadrupole mass spectrometer
Ionisation mode: EI (70 eV).
Source temperature: 250° C.
Transfer Line temperature: 250° C.
Full scanning between range m/z 25 to 450.
   Sample Preparation for GC-MS Analysis:

4 ml resin precursor was placed in 20 ml vial with a screwtop cap and a PTFE-lined silicone septum and incubated at 40° C. for 3 hours to equilibrate. Headspace sampling was performed by inserting a 1 cm Carboxen/Polydimethylsiloxane (75 μm) solid phase micro-extraction (SPME) fibre through the septum and into the headspace for 15 minutes, then de-sorbing the fibre in the GC injection port for 5 min.

Additionally, volatile analysis was performed by direct injection of up to 500 μl of headspace from the above incubation into the GC injection port using a gas-tight syringe.

Further analysis was performed by dissolving the resin in dichloromethane (50 mg/ml) and injecting 1 μl into the GC injection port.

For experiments using excess alcohol as the co-reactant/solvent, the re-condensed solvent following removal from the resin precursor was injected directly (1 μl) in the GC injection port.

Reactions and Results:

(i) Rapeseed oil in IPA FIG. 2 shows GC-MS results from ozonolysis of rapeseed oil in IPA. The results in FIG. 2a and FIG. 2b are from a time-course reaction with 500 g rapeseed oil and 2.51 IPA, with the temperature kept below 65° C. The reaction products were isolated after 30 min ozonization (FIG. 2a), ie. after incomplete ozonization, or after 270 min ozonization (FIG. 2b), ie. after complete (over) ozonization (201.2 g ozone used after 270 min). The results in FIG. 2c are from a reaction with 159.64 g rapeseed oil, 37.92 g IPA and 32 g ozone, with the temperature kept below 65° C.

Ozonization of rapeseed oil with ozone and reduced levels of IPA (FIG. 2c) gives a volatile profile, in terms of intensity and complexity, somewhere between incomplete ozonization in excess IPA (FIG. 2a), and complete (over) ozonization in excess IPA (FIG. 2b). In particular, the higher esters (isopropyl hexanoate and nonanoate) and free nonanoic acid become elevated with prolonged ozonization in excess alcohol.

(ii) Rapeseed Oil in Water/IMS and Water

FIG. 3 shows GC-MS results from ozonolysis of rapeseed oil in water/IMS and water. The results in FIG. 3a are from a reaction with 150.06 g rapeseed oil, 5.02 g water, 28.02 g IMS and 33.55 g ozone, with the temperature kept below 60° C. The results in FIG. 3b are from a reaction with 166.18 g rapeseed oil, 12.19 g water and 33.5 g ozone, with the temperature kept below 63° C.

Ozonization of rapeseed oil with these levels of both water and ozone (FIG. 3b), or water/IMS and ozone (FIG. 3a), gave the simplest and least intense volatile profiles, with the former showing no esters as might be predicted. The aroma of the resin precursors is entirely consistent with the observations made by GC-MS, with the over ozonated product in excess alcohol noticeably intense and pungent, but with optimised ozone and protic co-reactant, whether it is water, IMS or IPA, the aroma is vastly less intense.

NMR Analysis

NMR Parameters:

NMR spectra were recorded in CDCl₃ on a Bruker AC250 NMR spectrometer at 250 MHz for protons (128 scans) and 62.9 MHz for carbon (5000 scans) and in the latter case were broad-band decoupled.

Reactions and Results:

(i) Methyl Oleate in Excess IPA (with Additional GC-MS Analysis)

Methyl oleate standard was used as the substrate (purity 97%, contains 3% of methyl stearate). The methyl oleate (6.00 g, 20.0 mmol) was fully ozonated (until methyl oleate disappeared by TLC) with ozone (32 mmol, flow of oxygen 5 L/min) in excess of IPA (120 ml). Careful evaporation of IPA in vacuo (10 mmHg, 40° C.) afforded product (8.16 g) as a viscous oil. When 20 mmol of methyl oleate consumed 20 mmol of IPA and 20 mmol of ozone, we should expect 8.16 g of product.

Fractionation of the product mixture was performed on a Silica 60 gel column, eluted with ether/petrol ranging from 12:88 to 50:50. Various fractions were collected. Prior to further analysis by GC-MS, the collected fractions were diluted in dichloromethane.

Ozonolysis of methyl oleate in IPA leads, after solvent evaporation, to a viscous oil, ¹H and ¹³C NMR of which show that only a little amount of aldehydes (peaks at 9.76 ppm in ¹H and at 203 ppm in ¹³C NMR) and alcoxyhydroperoxides (peaks at 8.6 ppm in ¹H and at 106.16, 106.07 ppm in ¹³C NMR) are present (FIG. 4). The principal components of this reaction mixture are four peroxy hemi-acetals, which shows characteristic absorption at 4.80-5.25 ppm in ¹H NMR for methyne protons and 8 peaks in region from 100.67 to 105.61 ppm in ¹³C NMR for carbons, connected to a peroxide bridge.

In detail, the main components shown in FIG. 4 are four 1-(1-alkoxy-alk-1-ylperoxy)-alkan-1-ols (namely two diastereomers of 1-(1-isopropoxy-non-1-ylperoxy)-(8-methoxycarbonyl)-oct-1-ol and two diastereomers of 1-(1-isopropoxy-[8-methoxycarbonyl]-oct-1-ylperoxy)-nonan-1-ol), which present in ratio 1:1:1:1 (see FIG. 5, compounds 3 and 4). Also, it is possible to identify presence of small amount of two aldehydes and two 1-alkoxyalk-1-yl hydroperoxides (namely nonanal, (8-methoxycarbonyl)-oct-1-al, 1-isopropoxy-non-1-yl hydroperoxide, 1-isopropoxy-(8-methoxycarbonyl)-oct-1-yl hydroperoxide).

The chemical structures of these compounds are shown in FIG. 5. For each compound, the associated IUPAC formal name, and an alternative common name (in parentheses) are:
compound 1: 1-1-Isopropoxy-non-1-yl hydroperoxide (1-Isopropoxy-non-1-yl hydroperoxide);
compound 2: 9-Hydroperoxy-9-isopropoxy-nonanoic acid methyl ester (1-Isopropoxy-(8-methoxycarbonyl)-oct-1-yl hydroperoxide);
compound 3: 9-Hydroxy-9-(1-isopropoxy-nonylperoxy)-nonanoic acid methyl ester (1-(1-Isopropoxy-non-1-ylperoxy)-(8-methoxycarbonyl)-octan-1-ol);
compound 4: 9-(1-Hydroxy-nonylperoxy)-9-isopropoxy-nonanoic acid methyl ester (1-(1-Isopropoxy-[8-methoxycarbonyl]-oct-1-ylperoxy)-nonan-1-ol);
compound 5: 1-(1-Isopropoxy-non-1-ylperoxy)-nonan-1-ol (1-(1-Isopropoxy-non-1-ylperoxy)-nonan-1-ol); and
compound 6: 9-(1-Hydroxy-8-methoxycarbonyl-octylperoxy)-9-isopropoxy-nonanoic acid methyl ester (1-(1-Isopropoxy-[8-methoxycarbonyl]-oct-1-ylperoxy)-(8-methoxycarbonyl)-oct-1-ol).

Part of product (4.31 g) was dissolved in dichloromethane (40 ml) and extracted with sodium bicarbonate solution (0.84 g in 30 ml of water). Water phase was separated and 85% phosphoric acid (1.7 g) was added. This solution was extracted with dichloromethane (2×30 ml). After drying and solvent evaporating mixture of nonanoic acid and nonanedioic acid monomethyl ester (110 mg) in ratio 1:2. This corresponds 2% and 4% yield of these acids after ozonolysis.

Preparative column chromatography of the product mixture with removed acids (3.07 g) was performed on a Silica 60 gel column (100 g), eluted with ether/petrol ranging from 12:88 to 50:50. Various fractions were collected and analysed by ¹H, ¹³C NMR and GC-MS:
1) methyl stearate, 0.08 g,—impurity from starting material
2) nonanal, 0.34 g
3) 1-isopropoxy-non-1-yl hydroperoxide, 0.86 g
4) 1-isopropoxy-(8-methoxycarbonyl)-oct-1-yl hydroperoxide, 0.14 g
5) mixture, main component of which is 1-(1-isopropoxy-[8 methoxycarbonyl]-oct-1-ylperoxy)-(8-methoxycarbonyl)-oct-1-ol, 1.31 (8-methoxycarbonyl)-oct-1-al, 0.31 g.
   (ii) Methyl Oleate in IPA

Ozonization of methyl oleate in the presence of 1.5 mol.equiv. of IPA at 45° C. is shown in FIG. 6. NMRs of the reaction mixture show the following molar ratio of methyne carbons: secondary ozonides (signals A at 104.1 ppm, 13% (the same compounds as in (v) below), isopropoxyalkylhydroperoxides—(signals B at 106.0 ppm, 11%), hemiacetals of isopropoxyalkylhydroperoxides—(signals C at 100.6-101.2 and at 104.8-105.6 ppm, 67%), aldehydes 9%.

When this reaction was carried out in the presence of large excess of IPA at 20° C. (see (i) above and FIG. 4c), no secondary ozonides were detected.

(iii) Methyl Oleate in Water

Ozonization of methyl oleate at 45° C. in the presence of water (1.36 mol. equiv.) gave the following molar ratio of methyne carbons by ¹³C NMR (see FIG. 7): secondary ozonides (signals A at 104.1 ppm, 25%), hydroxyalkylhydroperoxides—(signals B at 101.4 ppm, 10%), hemiacetals of hydroxyalkylhydroperoxides—(signals C at 100.6-101.0 ppm, 54%). Molar ratio of aldehydes was found to be 11% by ¹H NMR.

(iv) Methyl Oleate in Excess Water

Ozonization of methyl oleate at 45° C. in the presence of water excess (5 mol.equiv.) led to a reaction mixture with the following product distribution (see FIG. 8): secondary ozonides (A, 16%), hydroxyalkylhydroperoxides—(B, 11%), hemiacetals of hydroxyalkylhydroperoxides—(C, 60%), aldehydes 13%. It is evident that quantity of secondary ozonides is lower in the present reaction compared with ozonization of methyl oleate at 45° C. in the presence of 1.36 mol.equiv. of water, where 25% secondary ozonides are formed (see (iii) above and FIG. 7).

(v) Methyl Oleate in a Large Excess of Water

Ozonization of methyl oleate at 45° C. in the presence of large excess of water (28 mol.equiv.) led to a reaction mixture with the following product distribution (see FIG. 9): secondary ozonides (A, 13%), hydroxyalkylhydroperoxides—(B, 10%), hemiacetals of hydroxyalkylhydroperoxides—(C, 64%) and aldehydes 13%.

(vi) Rape Seed Oil (RSO) in IPA

This sample was prepared for gas evaluation. 503.97 g (0.57 mole, 2.43 mole of unsaturation) of refined rapeseed oil was placed in a 1 litre reactor flask fitted with 4-necked lead and a overhead mechanical stirrer. 153.78 g (2.56 mole) of isopropyl alcohol (IPA) was added to the oil and mixed at high speed (around 300 rpm). stirring for 15 minutes. 158.60 g (3.30 mole) ozone was bubbled through the mixture at 0.61 g/minute (gas flow 5 litre per minute) over a period of 260 minutes. Starting and finishing temperatures were 8.1° C. and 49.7° C., respectively. The mixture was further stirred for 30 minutes to flush off residual ozone. Weight of final product was 646.40 g.

Ozonolysis of rape seed oil (RSO) in IPA leads to a viscous oil, ¹H and ¹³C NMR of which (FIG. 10) are very similar to those on FIG. 4 and show that only a little amount of aldehydes (peaks at 9.76 ppm in ¹H and at 203 ppm in ¹³C NMR) are present. The principal components of this reaction mixture are many peroxy hemi-acetals, which shows characteristic absorption at 5.1-5.4 ppm in ¹H NMR for methyne protons and many peaks in region from 100.0 to 106.5 ppm in ¹³C NMR for carbons, connected to a peroxide bridge.

(vii) Rape Seed Oil (RSO) in Water

150.06 g (0.17 mole, 0.69 mole of unsaturation) of refined rapeseed oil (iodine value 117.7) was mixed with 12.19 g (0.67 mole) water. The mixture was placed in a 500 ml reactor flask fitted with 4-necked lid and a overhead mechanical stirrer. Before introducing ozone, the mixture was stirred at high speed (around 300 rpm) for 15 minutes. Ozone was bubbled through the mixture at 0.61 g/minute (gas flow 5 litre per minute) for 55 minutes (total ozone 0.69 mole). Starting and finishing temperatures were 12.1° C. and 62.6° C., respectively. The reaction mixture was stirred for further 30 minutes to strip off residual ozone. Weight of final product was 190.60 g.

Ozonolysis of RSO in water leads to a viscous oil, ¹H NMR of which (FIG. 11) are very similar to that of FIG. 10 and show even smaller amounts of aldehydes (peaks at 9.65 ppm in ¹H and at 203 ppm in ¹³C NMR) are present. The principal components of this reaction mixture are many peroxy hemi-acetals, which shows characteristic absorption at 4.9-5.5 ppm in ¹H NMR for methyne protons.

HPLC Analysis

HPLC Parameters:

The HPLC column was a 25 cm×4.6 mm I.D. 5 μm Lichrospher RP18-5 endcapped reversed phase, operated at 1 ml/min eluent flow rate. Gradient separation starting with 60% aqueous methanol, programmed with linear gradient to 95% methanol at 8 minutes, followed by linear gradient to 100% methanol at 13 minutes, held for a further 12 minutes at 100% methanol. Formic acid modifier at 0.5% throughout.

Reactions and Results:

(i) CNSL in IPA/Methanol (with Additional GC-MS Analysis)

HPLC chromatograms of ozonized cashew nut shell liquid (CNSL) solutions (20 μl at 4 mg/ml) in isopropyl alcohol/methanol 1:1. UV detection at 275 nm (FIGS. 12a and 12c) and 254 nm (FIGS. 12b and 12d). Peaks 1 (6.8 minutes) and 2 (10.8 minutes) are each clusters of compounds that appear as a consequence of ozonization. Peaks 3 (17.4 minutes) and 4 (19.7 minutes) are clusters which are unsaturated compounds present in CNSL in the beginning but which are consumed as ozonization proceeds. The relative ratio's of the emerging peaks against the reducing peaks can be used to indicate the approximate state of reaction. We conclude that peak clusters 1 and 2 include the principal reaction products arising from the ozonization of CNSL. Chromatograms in FIGS. 12a and 12b show the separation of CNSL which has been ozonized only to approximately 50% of the desired end point, with peak clusters 3 and 4 still very evident. Chromatograms in FIGS. 12c and 12d show the separation of CNSL which has been fully ozonized.

Heat treatment of ozonized CNSL post ozonization leads to a reduction in the overall intensity of peak cluster 1, and an increase in peak cluster 2.

The monitoring of ozonization of CNSL by HPLC with UV detection at 254 nm is shown in FIG. 13. The x-axis represents the approximate % ozonization towards the desired end-point of reaction. Peaks 1 and 2 are actually clusters of compounds which appear as a consequence of ozonization. Peaks 3 and 4 are clusters which are compounds present at the start but which are consumed as ozonization proceeds. The relative ratio's of the emerging peaks against the reducing peaks can be used broadly to indicate the approximate state of reaction.

An alternative method for monitoring ozonization, i.e. by GS-MS, is shown in FIG. 14. GC-MS chromatograms are given for ozonized CNSL with ozonization only 25% complete (FIG. 14a), ozonization 50% complete (FIG. 14b) and ozonization complete (FIG. 14c). Peaks 2 and 3 represent unsaturated C7 aldehydes, and peak 1 is fully saturated C7 aldehyde (heptanal). As would be anticipated, the presence of unsaturated species declines with increased ozonization. (GC-MS conditions as stated previously).

Discussion

Some of the anticipated structures from the NMR analysis, including the peroxy hemi-acetal and hydroperoxy derivatives, were unstable at the GC injection port temperature of 250° C. The decomposition products were as predicted from the various different starting structures. These include nonanal, isopropyl nonanoate, nonanoic acid, octane, 9-oxo-nonanoic acid, 9-oxo nonanoic acid methyl ester, methyl stearate (impurity of starting material), nonanedioc acid (azelaic acid) and various mono/diesters thereof (see FIG. 2). This experiment served to demonstrate that GC-MS can be a useful tool for accelerating the decomposition of the peroxy hemi-acetal material to evaluate the VOCs which may be produced by the resin-precursor on prolonged storage.

In the presence of an aprotic solvent such as dichloromethane, the prior art tells us (see FIG. 1) that a secondary ozonide (3) is formed from the primary ozonide (reaction F), though with the subsequent addition of a protic solvent such as water, hydrolysis will lead to the formation of peroxy hemi-acetal products. In view of the asymmetric carbon atom at positions either side of the peroxy link in structure (4), numerous diastereomers are possible assuming a random recombination.

In the presence of heat and mild acid, the peroxy hemi-acetal (4) can cleave to yield an aldehyde and an alkoxyhydroperoxide (reaction E), as would be expected from the Criegee mechanism (reaction 1), and hence perform as a thermosetting resin.

Bond Strength

When using for example resorcinol as the nucleophile, and p-toluenesulphonic acid as the acid catalyst, the resin precursors (oils and CNSL) can be cured to a resin with a measured bond strength typically in the region of 5.2 to 6.5 mPa. This is the same range as for material prepared using excess co-reactant/solvent.

Co-Products

The use of excess ozone and excess protic co-reactant, commonly described in the prior art, leads to the generation of undesirable excess volatiles which increases the aroma intensity of the product markedly. The volatiles arise as a consequence of decomposition products of the product, as well as the action of ozone on the protic co-reactant/solvent. Compound classes generated in the presence of protic solvents, especially if used in excess, include:

1) Free fatty acids, including formic acid, acetic acid, hexanoic acid, heptanoic, octanoic and nonanoic acid. Heptanoic acid predominates in CNSL and nonanoic acid in vegetable oils, as a consequence of de-composition of the peroxy hemi-acetal. Stearic acid will be present in the vegetable oils to varying degrees due to rancidity, and also oleic, linoleic and linolenic acids if ozonolysis is incomplete.

2) Fatty acid esters as a consequence of both the breakdown of the hydroperoxy hemi-acetal where R is an alkyl moieity, and dependant upon the protic co-reactant (e.g. methyl for methanol, isopropyl for isopropanol etc), and also direct esterification of free fatty acids by the protic co-reactant when present at excess levels.

3) Aldehydes, such as nonanal, heptanal, hexanal, etc. as a consequence of cleavage of the primary ozonide without re-combination with the hydroperoxy ion. Also as a consequence of breakdown of alkyl hydroperoxide by homolytic scission, yielding either the aldehyde or an alkyl hydrocarbon depending upon the side of the oxygen that cleavage occurs. Unsaturated aldehydes such as 2-hexenal and 3-nonenal arise as a consequence of incomplete ozonolysis. Malonaldehyde is generated, though subsequently oxidised/esterified to esterified malonic acid, or further to smaller acids/esters and carbon dioxide, as discussed by Pryde et al. (1961; supra).

4) Acetals, as a consequence of reaction of aldehydes with excess protic co-reactant.

5) Alcohols—primarily from hydroxylated unsaturated fatty acyl chains in hydroxylated vegetable oils, and also alkyl phenolic alcohols such as cardol in CNSL when ozonolysis is incomplete.

6) Hydrocarbons, such as octane, arising potentially from both the homolytic scission of an alkyl hydroperoxide, or the peroxy hemi-acetal.

Heat treatment and over ozonation leads to the increased formation of octane, carbon dioxide, free fatty acids and esters (especially when the protic solvent is in excess), as a consequence of de-stabilisation of the peroxy hemi-acetal. Potentially, over ozonation could also lead to cleavage of the glyceryl ester bonds.

In the absence of sufficient protic co-reactant, secondary ozonide formation can occur. This can be avoided by ensuring adequate mixing of all the reactants.

Stabilisation by Heat Treatment—Analysis of Gasses Evolved

Stabilisation of the ozonization reaction product can be performed by heat treatment. This treatment brings about the thermal decomposition of the principal reaction products, such as 1-(1-alkoxyalk-1-yl peroxy)-alk-1-ol and 1-(1-hydroxyalk-1-yl peroxy)-alk-1-ol, and also free hydroperoxides and secondary ozonides. Heat treatment, in one step, liberates gas (O₂, CO₂) which would otherwise evolve from the reaction product medium on a slow and gradual basis at ambient. The heat-treated product contains a mixture of various aldehydes and carboxylic acids which can go on to perform as a thermosetting resin material. Problems encountered with gas liberation during formation of thermoset resin composites using non-heat-treated material can thus be avoided or diminished.

Examples of heat treatment of ozonolysis product and analysis of gases liberated are given below.

Ozonolysis product (80 g) resulting from ozonolysis of CNSL in water were placed into a 100 ml four neck round bottomed flask equipped with thermometer, gas inlet tube, mechanical stirrer and a small reflux condenser. The system's gas capacity, measured by the addition of water through the reflux condenser to fill the apparatus, was 43 ml. The system was flushed with nitrogen, the gas inlet closed and the condenser outlet connected via a narrow tube to a 250 ml trap bottle charged with a potassium pyrogallate solution (defined below). The outlet of the trap bottle was connected to an upturned 250 ml measuring cylinder filled with brine solution and resting in a 1 L beaker.

Vigorous stirring was started, then the reactor was heated by heating mantle to 80° C. when decomposition of the ozonization products from CNSL in water began. The heating mantle was turned off and the temperature of the reaction mixture spontaneously increased to 100° C. When the temperature began to drop, the mantle heating was introduced again to maintain the reaction at 100±2° C. After 1 h, the reactor was disconnected from the trap bottle.

The reagent in the trap bottle changed from colourless to dark brown, indicating that oxygen had been absorbed. The upturned cylinder contained 45 ml of gas (close to the system's gas capacity, indicating that all gas evolved was consumed by trap bottle). This suggested that the evolved gas contained oxygen, and, probably, carbon dioxide.

The trap bottle was flushed with nitrogen, then pyrogallol solution (8 g of pyrogallol in 25 ml of water) and potassium hydroxide solution (57 g of potassium hydroxide in 95 g of water) were introduced using a needle. (The trap bottle can absorb up to 1.5 L of oxygen and/or up to 12 L of carbon dioxide.)

In a separate experiment, ozonolysis product (80 g) resulting from ozonolysis of CNSL in water were placed into a 100 ml four neck round bottomed flask equipped with thermometer, gas inlet tube, mechanical stirrer and a small reflux condenser. The system was flushed with nitrogen then the gas inlet closed and the condenser outlet connected with a tube to an upturned 500 ml measuring cylinder filled with 0.1 M barium hydroxide solution and sitting in a 1 L beaker. With vigorous stirring, the reactor was heated by the heating mantle to 80° C. when decomposition of the ozonolysis product from CNSL in water began. The heating was stopped and the reaction mixture spontaneously heated to 100° C. When temperature began to drop, mantle heating was introduced to keep maintain the reaction at 100±2° C. After 1 h, the reactor was disconnected from the upturned measuring cylinder which contained 465 ml of gas (of which 43 ml was nitrogen and 422 ml oxygen). A precipitate of barium carbonate, formed by reaction of carbon dioxide with barium hydroxide in the cylinder, was filtered and titrated with 0.5M hydrochloric acid solution to show that 14.1 mmol of carbon dioxide (340 ml at 20° C.) was evolved.

Thus, degassing of 80 g of CNSL ozonized in water gives 422 ml of oxygen and 340 ml of carbon dioxide. This corresponds to formation of 9.5 L of gas per 1 kg of ozonized CNSL (gas contains 45% CO₂ and 55% O₂).

The same procedure was applied to analysing degassing of ozonolysis reaction product from rapeseed oil (RSO) ozonized in water, except that decomposition started at 90° C. and the temperature spontaneously rose to 110° C., whereafter it was kept at 100±2° C. The result was that 4.7 L of gas per kg of ozonized RSO was evolved. The gas contained 39% CO₂ and 61% O₂.

At lower temperatures, de-gassing occurs very slowly and under these circumstances, for example, gas collected over ozonized CNSL in water at 50° C. contains 23% CO₂ while gas collected over ozonized RSO at 60° C. contains 27% CO₂.

Determination of Ozonization Exotherm (ΔH) for Alkenes

For engineering and process considerations, the exotherm from ozonization needs to be understood.

During the ozonization reaction the following bonds need to be destroyed: (a) carbon-carbon double bond (bond energy in range 610-630 kJ/mol); (b) ozone oxygen-oxygen bonds (bond energy 605 kJ/mol); (c) oxygen-hydrogen bonds (bond energy in the range 460-464 kJ/mol).

After ozonization, 6 new bonds are formed:
(a) 4 carbon-oxygen bonds (bond energy in the range 355-380 kJ/mol); (b) peroxide oxygen-oxygen bond (bond energy ca 155 kJ/mol); (c) oxygen-hydrogen bond (bond energy in the range 460-464 kJ/mol).
Minimal energy from ozonization: ΔHᵣ=−(4×355+155−630−605)=−340 kJ/mol
Maximal energy from ozonization: ΔHᵣ−(4×380+155−610−605)=−460 kJ/mol.
   Determination of Exotherm (ΔH) for Rapeseed Oil (RSO) Ozonization

RSO (253.0 g) and IPA (70.7 g, 1.18 mol, 1.1 mol.equiv. to C═C) were placed in a Pyrex flask (380.1 g) with thermometer probe, gas inlet, mechanical stirrer (300 RPM) and condenser with gas outlet. The flask was put into a Dewar vessel with a flat magnetic stirrer and with good isolation of inside volume from atmosphere. The temperature inside was 12.2° C. before water addition. Another Dewar vessel was filled with warm water (1,002 g). When the temperature equilibrated (30.15° C.) the water was poured quickly into the first Dewar vessel. The level of the water layer was adjusted to the level of the reaction mixture with a lab jack. Within 15 minutes the water and organic phase reached a thermodynamic equilibrium at 24.8° C. The water temperature dropped by 30.15−24.8=5.35° C. The complete system continued to cool down at ca 0.06° C./minute. After 15 minutes, which was necessary to reach a thermodynamic equilibrium, the system cooled by 15×0.06=0.9° C. under ambient conditions. The corrected drop of water temperature is 5.35−0.9=4.45 ° C.

The heat capacity (Cₚ) of water is 4.184 Jg⁻¹K⁻¹. The heat consumed by the glassware and the reaction mixture is 4.45×4.184×1002=18.66 kJ. The temperature of the glassware and the reaction mixture was raised by 24.8−12.2=12.6° C. Therefore the Cₚ (glassware and reaction mixture)=18.66/12.6=1.48 kJK⁻¹. When warm water was replaced with cold water (1,000 g) a new thermodynamic equilibrium was reached at 7.2° C. The gas outlet was connected to an ozone trap with buffered potassium iodide. At t=0 min, ozone was introduced at a constant rate of 9.2 nmol/min (oxygen flow 5 L/min). After 20 min the ozone generator was stopped and oxygen flow stopped. The ozone escape was 8.1 mmol. Therefore, ozone consumption was 20×9.2−8.1=175.9 mmol. 16 minutes after stopping the reaction the system reached a thermodynamic equilibrium at 17.8° C. The temperature of the system was raised by 17.8−7.2=10.6 ° C. The Cₚ (all system) is 1.48+4.184×1.000=5.66 kJK⁻¹. The heat generated by the reaction is 5.66×10.6=60.0 kJ.

The uncorrected ozonization is ΔH−60.0/0.1759=−341 kJ/mol. The corrected ozonization, allowing for heat arising from the flow of oxygen and evaporation of IPA ΔH is −394 kJ/mol.

The determination of ozonization exotherm as above is useful for industrial applications because knowing the amount of heat generated by a reaction allows for control of the reaction.

Process Methodology

Specific Examples of Process Methodologies:

### EXAMPLE 1

### Simulating Prior Art Method

Batch process in an excess of solvent—203.0 g of refined rapeseed oil was weighed into a 2,000 ml round bottom flask with 1,000 ml of iso-propanol. A mixture of ozone and oxygen were bubbled through the liquid for 180 minutes at a rate of 5 lmin⁻¹. The ozone content of the gas stream was 0.6 gmin⁻¹. The reaction mixture was continuously stirred.

The temperature of the reaction mixture was maintained at 15° C.±2° C. by resting the reaction flask in an ice/water bath.

The reaction mixture whilst initially cloudy produced a clear colourless liquid product. The excess alcohol was removed from the product by rotary evaporation under vacuum. The product exhibited a strong, persistent, sharp fruity odour.

Headspace analysis of the volatile co-products indicated an intense profile of compounds including esters, acids, acetals, aldehydes, hydrocarbons typically produced by the mechanisms outlined above.

### EXAMPLE 2

Batch process in a reduced volume of solvent—151.0 g of refined rapeseed oil was weighed into a 500 ml round bottom flask with 36.0 g of iso-propanol. A mixture of ozone and oxygen were bubbled through the liquid for 50 minutes at a rate of 5 lmin⁻¹. The ozone content of the gas stream was 0.6 gmin⁻¹. The reaction mixture was continuously stirred.

The temperature of the reaction mixture was allowed to rise from 10° C. to 60° C. over the first 20 minutes of the reaction, there after the temperature was maintained at 60° C.±2° C. by resting the reaction flask in a cold water bath.

The ozone content of the off-gas was measured intermittently during the course of the reaction rising from around 5% for the first 40 minutes of the reaction to 10% at the end.

The reaction mixture whilst initially cloudy produced a clear pale straw yellow liquid product. The product exhibited a much milder fruity odour.

Headspace analysis of the volatile co-products indicated the presence of the low molecular weight esters and other compounds but in much smaller quantities than when excesses of alcohol and ozone were used.

The weight of final product was 182.7 g giving a yield of 84%.

### EXAMPLE 3

Batch process in a reduced volume of solvent (water as participating solvent/reactant)—200.3 g of CNSL were weighed into a 500 ml round bottom flask with 28.1 g of water. A mixture of ozone and oxygen were bubbled through the liquid for 120 minutes at a rate of 5 lmin⁻¹.

The ozone content of the gas stream was 0.6 gmin⁻¹. The reaction mixture was continuously stirred.

The temperature of the reaction mixture was allowed to rise from 10° C. to 60° C. over the first 35 minutes of the reaction, there after the temperature was maintained at 60° C.±2° C. by resting the reaction flask in a cold water bath.

The ozone content of the off-gas was measured intermittently during the course of the reaction rising from around 3% for the first 100 minutes of the reaction to 15% at the end.

The reaction mixture produced an opaque brown liquid product. The odour of the final product was similar to the starting material and headspace analysis of the material was shown to be free from many of the malodorous compounds associated with the alcohol systems. The weight of final product was 182.7 g giving a yield of 80%.

### EXAMPLE 4

Continuous process with a reduced volume of solvent—A metered intimate mixture of five parts refined soybean oil to one part industrial methylated spirit were sprayed at a constant rate of 3.1 gmin⁻1 from the top of a reaction chamber. The reaction chamber contained concentric tubes on which the liquid spray formed a thin falling film thus providing a large reaction surface area. A gas mixture of ozone and oxygen was continuously pumped into the chamber. The inlet gas contained 0.1 gl⁻¹ ozone. The gas flow through the chamber was regulated to around 5 lmin⁻¹ by measurement of the ozone content of the off-gas and loop control to the ozone generator input.

The product, a clear colourless liquid, was collected from a drainpipe at the base of the reaction chamber. The product exhibited a mild fruity odour.

The reaction chamber was cooled by means of a counter current cold water coil maintaining a temperature of 50° C.±5° C.

General Comments on Process Methodologies:

The resin pre-cursor product can be derived directly from a batch or continuous process involving the reaction of a concentration of ozone in air or oxygen with a mixture of vegetable oil(s) or CNSL and a participating co-reactant.

Intimate contact of the reactants can be achieved by well-known forms of reactor. Reactor designs particularly suitable as those provided in the FIGS. 15 to 19.

In FIG. 15, a vegetable oil and co-reactant mixture (1) is transferred by means of a pump (2) into a continuous reaction vessel (3) containing concentric tubes to maximize the reaction surface area. The oil and co-reactant mixture is sprayed into the vessel to provide a thin film coverage of the surfaces. Ozone enters the vessel from an ozone generator. Resin precursor product (4) is removed from the vessel, while gaseous products exit via an off-gas outlet.

In FIG. 16, a vegetable oil and co-reactant mixture (1) is transferred by means of a pump (2) into a continuous reaction vessel (3) containing a helix structure. The oil and co-reactant mixture is metered into the vessel to provide a thin film coating of the helix. Ozone enters the vessel from an ozone generator. Resin precursor product (4) is removed from the vessel, while gaseous products exit via an off-gas outlet.

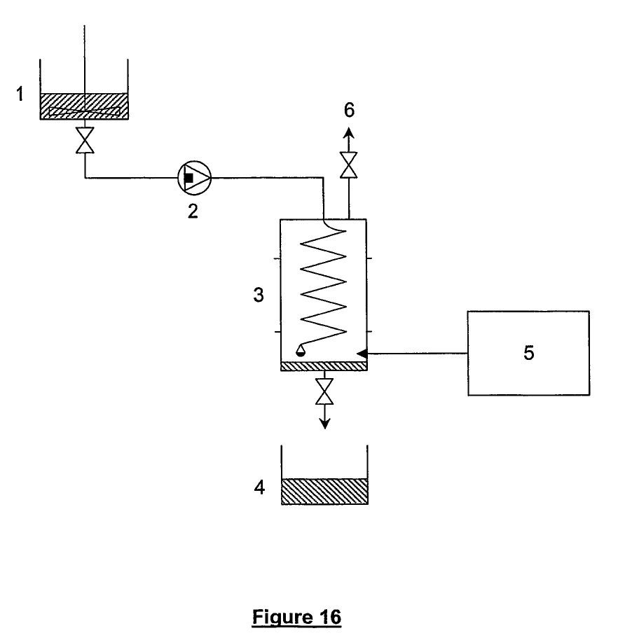

In FIG. 17, a vegetable oil and co-reactant mixture (1) is transferred by means of a pump (2) into a continuous reaction vessel (3) packed with glass spheres to maximise the reaction surface area. The oil and co-reactant mixture is sprayed into the vessel to provide a thin film coverage of the surfaces. Ozone enters the vessel from an ozone generator. Resin precursor product (4) is removed from the vessel, while gaseous products exit via an off-gas outlet.

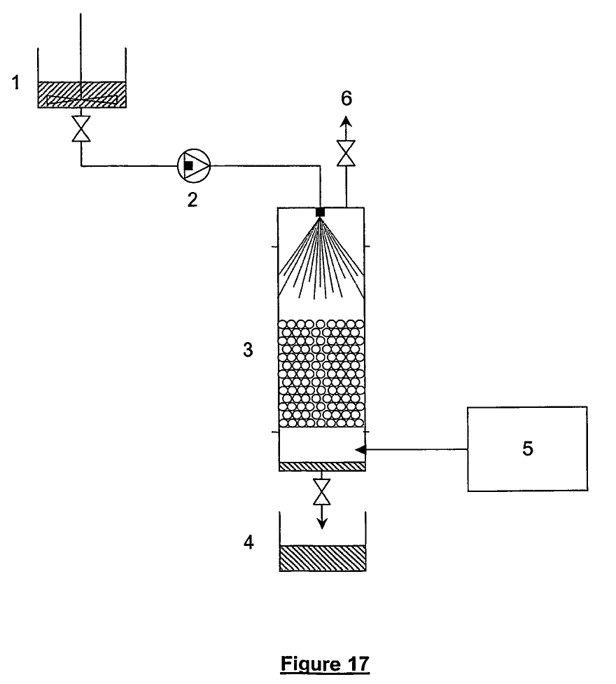

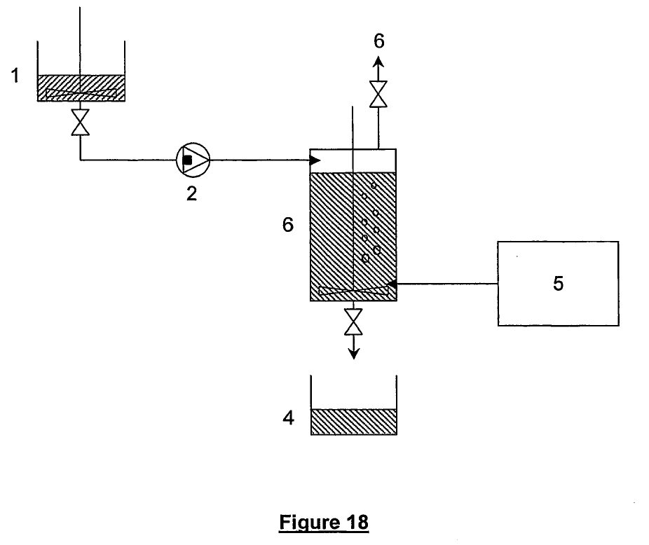

In FIG. 18, a vegetable oil and co-reactant mixture (1) is transferred by means of a pump (2) into a batch reaction vessel (6). Ozone from an ozone generator is bubbled through a stirred oil and co-reactant mixture in the vessel. Resin precursor product (4) is removed from the vessel, while gaseous products exit via an off-gas outlet.

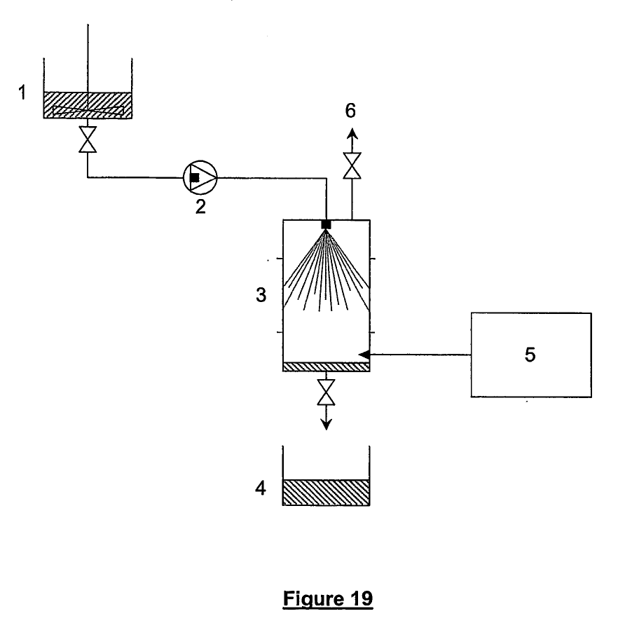

In FIG. 19, a vegetable oil and co-reactant mixture (1) is transferred by means of a pump (2) into a continuous reaction vessel (3). The oil and co-reactant mixture is sprayed into the vessel to provide a thin film coverage of the surfaces. Ozone enters the vessel from an ozone generator. Resin precursor product (4) is removed from the vessel, while gaseous products exit via an off-gas outlet.

Based on the rate of consumption of ozone, the reaction is virtually instantaneous.

The product depending on the reaction components is collected from the reaction vessel as a clear or opaque liquid, which is colourless or pale yellow in the case of vegetable oils or brown from CNSL.

Yields from the process are typically around 90% for an oil/water system and 75% for an oil/alcohol mixture.

Excess heat generated by the exothermic reaction can be removed as necessary from the reactor and/or reaction medium by counter current liquid or gas flow or any other conventional method.

The concentration of the ozone in the air or oxygen is a function of the efficiency of the ozone generator, typically but not limited to 1 to 15% by weight.

Key features and benefits of the process are:

1. Ambient/Elevated Reaction Temperature.

Operating at higher temperatures than previously reported in the art is possible owing to the lower levels of solvent which otherwise would result in un-desirable co-products.

2. Improved Processability/Mass Transfer.

It is customary in the art to use excess solvent as a diluent to facilitate the reaction and mass transfer. This is particularly important as the viscosity of the reacting medium increases typically 40-fold during processing. When operating at reduced temperatures, this is particularly troublesome.

Adopting ambient or elevated reaction temperatures reduces the product viscosity without the need for excess solvent addition.

3. Lower Levels of Co-Products.

As an environmental requirement, co-products released during manufacture must be eliminated by appropriate, expensive plant design. Those that remain in the product are un-desirable as they produce a persistent and irritating odour that reduces the commercial value.

Production of co-products also consumes valuable ozone requiring additional capital and revenue expenditure, and reduces the product yield.

4. Rapid Process Kinetics.

Allows process flexibility ranging from large-scale batch processing to a small volume, high-throughput continuous process.

5. Low or Solvent-Free Process.

Use of solvent is un-desirable due cost, safety and environmental issues. Bulk handling of solvent requires higher capital investment to remove flash points, requires intrinsically safe electrical systems and additional safety systems.

Furthermore, spent solvent must be recovered and re-cycled or disposed of through specialist routes.

1. A process for the ozonolysis of unsaturated oils to form ozonolysis reaction products, comprising reacting together
(a) ozone,
(b) unsaturated oils, and
(c) a participating co-reactant,
wherein the participating co-reactant is present in 0.01 to less than 1 part by mass per part of the unsaturated oil.

2. The process of claim 1, wherein the ozonolysis reaction products essentially comprise peroxy hemi-acetals.

3. The process of claim 1 wherein the participating co-reactant comprises water.

4. The process of claim 3, wherein the participating co-reactant is a mixture of water.

5. The process claim 4, comprising introducing the ozone into a reactor vessel containing a mixture comprising the unsaturated oil and the participating co-reactant.

6. The process of claim 5, wherein the process is conducted at a temperature of −5° C. to 100° C.

7. The process of claim 4, comprising introducing into a reactor vessel containing the unsaturated oil a vapor stream comprising the participating co-reactant and the ozone.

8. The process of claim 7, wherein the process is conducted at a temperature of 10° C. to 140° C.

9. The process of claim 4, comprising introducing into a reactor vessel containing the ozone a mixture comprising the unsaturated oil and the participating co-reactant.

10. The process of claim 9, wherein the process is conducted at a temperature of −5° C. to 100° C.

11. The process of claim 4, comprising introducing separately into a reactor vessel:
(a) a spray comprising the unsaturated oil;
(b) the ozone; and
(c) a vapor stream comprising the participating co-reactant.

12. The process of claim 11, wherein the process is conducted at a temperature of 70° C. to 140° C.

13. The process of claim 12, wherein the process is a batch process.

14. The process of claim 13, wherein the ozone is present in 0.1 to 0.6 parts by mass per part of unsaturated oil and participating co-reactant.

15. The process of claim 14, wherein the ozone is present at a concentration of 1 to 15% by mass in a mixture with air or oxygen.

16. The process of claim 15, wherein the unsaturated oil comprises plant oil.

17. The process of claim 15, wherein the unsaturated oil comprises animal oil.

18. The process of claim 17, further comprising a heat-treating step following ozonolysis.

19. The process of claim 18, wherein the ozonolysis reaction products are degassed.

20. The process of claim 19, wherein the heat-treating step involves heating to about 80° C. or above.

21. A process for making an adhesive-forming compound comprising treating under reducing conditions the ozonolysis reaction products produced by the process of claim 20.

22. A process for forming an adhesive material, comprising treating with an acidic material the adhesive-forming compound produced by the process of claim 21.

23. A process for forming an adhesive material, comprising treating with a basic material the adhesive-forming compound produced by the process of claim 21.

24. The process of claim 23, further comprising a heat-treating step.

25. A process for forming an adhesive material, comprising heat-treating the adhesive-forming compound produced by the process of claim 21.

26. The process of claim 25 wherein the adhesive is a resin.

27. A resin produced by the process of claim 26.

28. A resin derived from a product produced by the process of claim 26.

29. The resin of claim 28, wherein the resin is a cured thermosetting resin.

30. A solid composite material comprising the resin of claim 29.

31. An apparatus for performing the process of claim 26, comprising a reactor for formation of a resin precursor.

32. The process of claim 2, wherein the peroxy hemi-acetals are 1-(1-alkoxyalk-1-yl peroxy)-alk-1-ol.

33. The process of claim 2, wherein the peroxy hemi-acetals are 1-(1-hydroxyalk-1-yl peroxy)-alk-1-ol.

34. The process of claim 1, wherein the participating co-reactant comprises an alcohol.

35. The process of claim 1, wherein the participating co-reactant comprises water and an alcohol.

36. The process of claim 34, wherein the alcohol is selected from the group consisting of ethanol, industrial methylated spirits, and isopropanol.

37. The process of claim 35, wherein the alcohol is selected from the group consisting of ethanol, industrial methylated spirits, and isopropanol.

38. The process of claim 2, wherein the participating co-reactant comprises water.

39. The process of claim 2, wherein the participating co-reactant comprises an alcohol.

40. The process of claim 2, wherein the participating co-reactant comprises water and an alcohol.

41. The process of claim 39, wherein the alcohol is selected from the group consisting of ethanol, industrial methylated spirits, and isopropanol.

42. The process of claim 40, wherein the alcohol is selected from the group consisting of ethanol, industrial methylated spirits, and isopropanol.

43. The process claim 3, wherein the participating co-reactant is an alcohol.

44. The process of claim 43, wherein the alcohol is selected from the group consisting of ethanol, industrial methylated spirits, and isopropanol.

45. The process of claim 6, wherein the process is conducted at a temperature of 15° C. to 60° C.

46. The process of claim 10, wherein the process is conducted at a temperature of 15° C. to 50° C.

47. The process of claim 12, wherein the process is a continuous process.

48. The process of claim 16, wherein the plant oil is selected from one or more of the following group consisting of: cashew nut shell liquid, oil seed rape, linseed, soya, olive oil, castor oil, mustard seed oil, and ground nut oil.

49. The process of claim 17, wherein the animal oil is a fish oil.

50. The process of claim 17, wherein the animal oil is a fractionated tallow.

51. The process of claim 17, wherein the animal oil comprises fish oil and a fractionated tallow.

52. The process of claim 20, wherein the heat-treating step involves heating to about 80° to 125° C.

53. The process of claim 52, wherein the heat-treating step involves heating to about 80° C. to 110° C.

54. The process of claim 53, wherein the heat-treating step involves heating to about 80° C. to 100° C.

55. The process of claim 54, wherein the heat-treating step involves heating to about 80° C. to 90° C.

56. The process of claim 18, wherein the heat-treating step involves heating to about 80° C. or above.

57. The process of claim 56, wherein the heat-treating step involves heating to about 80° to 125° C.

58. The process of claim 57, wherein the heat-treating step involves heating to about 80° C. to 110° C.

59. The process of claim 58, wherein the heat-treating step involves heating to about 80° C. to 100° C.

60. The process of claim 59, wherein the heat-treating step involves heating to about 80° C. to 90° C.

61. The process of claim 21, wherein the adhesive-forming compound is an aldehyde.

62. The process of claim 22, further comprising a heat-treating step.

63. The resin of claim 27, wherein the resin is a cured thermosetting resin.

64. A solid composite material comprising the resin of claim 28.
